(19) United States
(12) Patent Application Publication
(10) Pub. No.: US 2024/0270729 A1
(43) Pub. Date: Aug. 15, 2024

(54) (THI)OXAZOLINE PESTICIDES

(71) Applicants: ELANCO TIERGESUNDHEIT AG, Basel (CH); BAYER ANIMAL HEALTH GMBH, Leverkusen (DE)

(72) Inventors: Pierre Ducray, Greenfield, IN (US); Denise Rageot, Greenfield, IN (US); Andreas Turberg, Greenfield, IN (US)

(21) Appl. No.: 18/569,370

(22) PCT Filed: Jun. 15, 2022
(86) PCT No.: PCT/EP2022/066343

§ 371 Date: Dec. 12, 2023

Related U.S. Application Data
(60) Provisional application No. 63/211,486, filed on Jun. 16, 2021.

(51) Int. Cl. C07D 413/04; A61K 31/4439; A61P 33/14; C07D 413/14

(52) U.S. Cl. CPC C07D 413/04; A61K 31/4439; A61P 33/14; C07D 413/14

(57) ABSTRACT

The present invention provides compounds of formula (I):

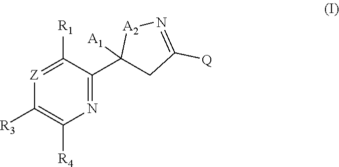

which are useful for long-lasting treatment and control of pests, optionally fleas and ticks, in companion animals and livestock, and pharmaceutical compositions and methods of using the same.

### FIELD

The present invention relates to medicinal chemistry, pharmacology, and veterinary and human medicine.

### BACKGROUND

The heteroaryl isoxazolines are used in agriculture, forestry, turf, household, wood products, nursery crops protection, and veterinary fields. The veterinary field includes companion animals and livestock, including fish. For example, such inhibitors are disclosed in WO 2005/085216, WO 2007/079162, US 2007/066617, US20130131017, WO 2009/002809, WO 2009/112275, WO 2010/003923, WO 2010/070068, WO 2012/120399, WO 2013/079407, and WO 2021/127188.

In many applications, long lasting effect against pests is desirable. Long lasting protection is particularly important with companion animals, such as dogs and cats and also mice, guinea pigs, ferrets, and rabbits; and with ranched animals such as cattle, sheep, pigs, and fish, in particular salmon and sea bass.

### SUMMARY

The present invention relates to a compound of formula (I) having extended half-life in companion animals and livestock, in particular, warm-blooded animals, especially dogs, cats and cattle, and their use in the control of ectoparasites. In many cases the compound of formula (I) provides long duration of action for months after a single oral administration or an injection.

The present invention also provides compounds of formula (I) which effectively treat and/or control ectoparasites in companion animals and livestock.

In one embodiment, the present invention provides a compound of formula (I):

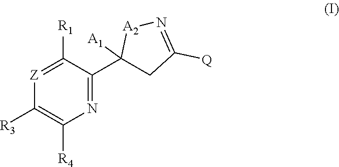

wherein
   A₁ is selected from the group consisting of CF₃, CHF₂, CH₂F, and CF₂CF₃;
   A₂ is O or S;
   Z is N or CR₂, preferably CR₂;
   R₁ is selected from the group consisting of hydrogen and halogen;
   R₂ is selected from the group consisting of hydrogen, halogen, difluoromethyl, trifluoromethyl, and trifluoromethoxy; preferably selected from the group consisting of halogen, difluoromethyl, and trifluoromethyl; more preferably selected from the group consisting of fluorine, chlorine, difluoromethyl, and trifluoromethyl;
   R₃ is selected from the group consisting of hydrogen, halogen, and trifluoromethyl;
   R₄ is selected from the group consisting of hydrogen, halogen, difluoromethyl, trifluoromethyl, hydroxy, methoxy, and trifluoromethoxy; preferably selected from the group consisting of halogen, difluoromethyl, and trifluoromethyl; more preferably selected from the group consisting of fluorine, chlorine, difluoromethyl, and trifluoromethyl;
   Q is selected from the group consisting of

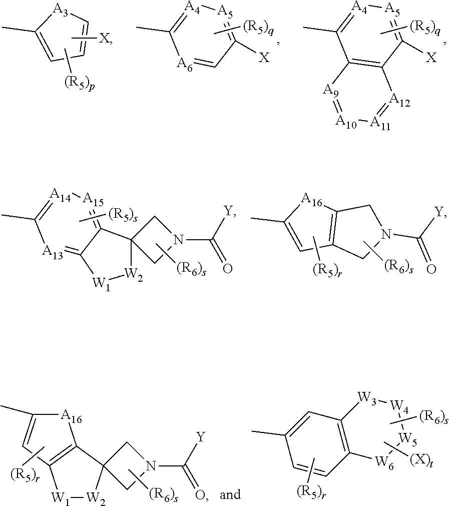

wherein
   p is 0, 1, or 2;
   q is 0, 1, 2, or 3;
   r is 0 or 1;
   s is 0, 1, or 2;
   t is 0 or 1;
   R₅, at each occurrence, is independently selected from the group consisting of halogen; cyano; nitro; hydroxyl; —NH₂; —NH(C₁-C₄ alkyl); —N(C₁-C₄ alkyl)₂; C₂-C₅-alkoxycarbonyl; C₁-C₆-alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl; C₁-C₆-alkoxy optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl; —NR₇C(O)(C₁-C₄ alkyl) optionally substituted on the C₁-C₄ alkyl with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂, wherein R₇ is independently selected from the group consisting of hydrogen and C₁-C₄ alkyl; —C(O)NR₇(C₁-C₄ alkyl) optionally substituted on the C₁-C₄ alkyl with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂, wherein R₇ is independently selected from the group consisting of hydrogen and C₁-C₄ alkyl; —SC₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂; and —S(O)C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂;
   R₆, at each occurrence, is independently selected from the group consisting of oxo, C₁-C₄ alkyl, and C₃-C₆ cycloalkyl;
   A₃ is O or S;
   A₄ is CH or N;
   A₅ is CH or N;
   A₆ is CH or N;
   A₇ is CHO, S, a bond, or N;
   A₈ is CH O, S, a bond, or N;
   A₉ is CH or N;
   A₁₀ is CH or N;
   A₁₁ is CH or N;
   A₁₂ is CH or N;
   A₁₃ is CH or N;
   A₁₄ is CH or N;
   A₁₅ is CH or N;
   A₁₆ is NR, O, or S, wherein R is selected from the group consisting of hydrogen, C₁-C₄ alkyl, and C₃-C₆ cycloalkyl;
   W₁ is selected from the group consisting of —O—, —S—, —NR₈—, —NC(O)R₉—, —CH₂—, and —C(O)—;
   W₂ is selected from the group consisting of —O—, —S—, —NR₈—, —NC(O)R₉—, —CH₂—, and —C(O)—;
   provided that:
      when W₁ is —O—, —S—, —NR₈—, or —NC(O)R₉— then W₂ is —CH₂— or ——C(O)—; and
      when W₂ is —O—, —S—, —NR₈—, or —NC(O)R₉— then W₁ is —CH₂— or ——C(O)—;
   W₃ is selected from the group consisting of nil, —O—, —S—, —S(O)—, —S(O)₂—, —NR₈—, —CH—, —N—, —CH₂—, and ——C(O)—;
   W₄ is selected from the group consisting of nil, —O—, —S—, —S(O)—, —S(O)₂—, —NR₈—, —CH—, —N—, —CH₂—, and ——C(O)—;
   W₅ is selected from the group consisting of nil, —O—, —S—, —S(O)—, —S(O)₂—, —NR₈—, —CH—, —N—, —CH₂—, and ——C(O)—;
   W₆ is selected from the group consisting of nil, —O—, —S—, —S(O)—, —S(O)₂—, —NR₈—, —CH—, —N—, —CH₂—, and ——C(O)—;
   wherein the bonds between W₁, W₂, W₃, and W₄ may be single or double bonds; provided that:
      (i) not more than two of W₁, W₂, W₃, and W₄ are nil;
      (ii) not more than two of W₁, W₂, W₃, and W₄ are —O—, —S—, —S(O)—, —S(O)₂—, —NR₈—, or ——C(O)—;
      (iii) if two of W₁, W₂, W₃, and W₄ are —O— and/or —S— then at least one carbon atom is present between them; and
      (iv) when W₁, W₂, W₃, or W₄ is —CH— and/or —NR₈— then a double bond is formed within the ring formed by W₁, W₂, W₃, and W₄;
   R₈, at each occurrence, is independently selected from the group consisting of hydrogen and C₁-C₆-alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl;
   R₉, at each occurrence, is independently selected from the group consisting of oxo, C₁-C₄ alkyl, and C₃-C₆ cycloalkyl;
   X is 5- to 10-membered heteroaryl having 1 or 2 heteroatoms selected from the group O, S, and N,
      wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, —C(O)NH—C₃-C₆ cycloalkyl, —C(O)NH—C₁-C₆ alkyl, and —C(O)NH—C₁-C₆ haloalkyl,
      wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, and C₃-C₆ cycloalkyl; or
   X is selected from the group consisting of

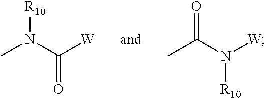

wherein
R₁₀ is selected from the group consisting of hydrogen, C₁-C₄ alkyl, C₁-C₄ haloalkyl, C₃-C₆ cycloalkyl, C₄-C₇ alkylcycloalkyl, C₂-C₇ alkylcarbonyl, C₂-C₅ alkoxycarbonyl, C₂-C₆ alkenyl, and C₂-C₆ alkynyl;
W is selected from the group consisting of
(i) hydrogen;
(ii) C₁-C₆ alkyl optionally substituted with 1 to 5 substituents independently selected from the group consisting of halogen; cyano; hydroxyl; oxo; C₁-C₄ alkoxy; C₃-C₆ cycloalkyl optionally substituted by 1 to 3 substituents independently selected from the group halogen and cyano; acetylenyl; —NH₂; C₁-C₇ aminocarbonyl; —NH(C₁-C₄ alkyl); —N(C₁-C₄ alkyl)₂; —SC₁-C₄ alkyl; —S(O)C₁-C₄ alkyl; —SO₂C₁-C₄ alkyl; —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, C₃-C₆ cycloalkyl, and —NH₂; —C(O)NH—C₁-C₆ alkyl optionally substituted with 1 to 5 substituents independently selected from the group consisting of halogen, cyano, hydroxyl, C₁-C₄ alkoxy, C₃-C₆ cycloalkyl, and —NH₂; —C(O)NH—C₁-C₆ cyanoalkyl optionally substituted with 1 to 3 halogen; —C(O)NH—C₁-C₆ haloalkyl; —C(O)-4- to 7-membered heterocycloalkyl attached by a nitrogen and optionally having 1 or 2 other heteroatoms selected from the group O, S, and N, wherein the carbons of the 4- to 7-membered heterocycloalkyl are optionally substituted with 1 to 4 substituents independently selected from the group consisting of halogen, cyano, nitro, hydroxyl, oxo, —NH₂, C₁-C₇ aminocarbonyl, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any other N in the 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, and C₁-C₄ alkyl optionally substituted with 1 to 5 substituents independently selected from the group consisting of halogen, cyano, hydroxyl, acetylenyl, C₁-C₄ alkoxy, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl; 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, C₃-C₆ cycloalkyl, C₁-C₄ haloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any S in the heteroaryl is optionally substituted with 1 or 2 oxygen atom(s); phenyl optionally substituted with 1 to 3 substituents selected from the group consisting of halogen, C₁-C₄ alkyl, cyano, and hydroxyl; C₃-C₆ cycloalkyl optionally substituted with 1 to 5 substituents independently selected from the group consisting of halogen, cyano, hydroxyl, oxo, C₁-C₄ alkoxy, C₁-C₄ alkyl optionally substituted with 1 to 3 groups selected from the group consisting of halogen and cyano, C₁-C₄ haloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, —C(O)NH—C₁-C₆ haloalkyl, C₂-C₆ alkenyl, and C₂-C₆ alkynyl; and 4- to 7-membered heterocycloalkyl having 1 or 2 heteroatoms selected from the group O, S, B, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, and C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, wherein any B in the of the 4- to 7-membered heterocycloalkyl or optionally benzo-fused 4- to 7-membered heterocycloalkyl, valency permitting, is substituted with hydroxyl, wherein any N in the 4- to 7-membered heterocycloalkyl or optionally benzo-fused 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, —C(O)—NH₂, C₁-C₄ alkyl optionally substituted with 1 to 5 substituents independently selected from the group consisting of halogen, cyano, hydroxyl, acetylenyl, C₁-C₄ alkoxy, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ haloalkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s);
(iii) C₃-C₆ cycloalkyl optionally substituted with 1 to 5 substituents independently selected from the group consisting of halogen, cyano, hydroxyl, oxo, carboxyl, C₁-C₄ alkoxy, C₁-C₄ alkyl optionally substituted with 1 to 3 groups selected from the group consisting of halogen and cyano, C₁-C₄ haloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, —C(O)NH—C₁-C₆ haloalkyl, C₂-C₆ alkenyl optionally substituted with 1 to 3 halogens, and C₂-C₆ alkynyl;
(iv) 6-membered aryl or 5- to 10-membered heteroaryl having 1, 2 or 3 heteroatoms selected from the group O, S, and N, wherein the carbons of the 6-membered aryl and the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl, wherein any N in the heteroaryl, valency permitting, is optionally substituted with a substituent selected from the group consisting of hydrogen, and C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl;
(v) 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkoxy, C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, —C(O)—NH₂, C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —SO₂NH(C₁-C₄ alkyl), —SO₂N(C₁-C₄ alkyl)₂, —C(O)NH—C₃-C₆ cycloalkyl, and —C(O)NH—C₁-C₆ haloalkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s); and
(vi) —NR₁₁R₁₂
wherein
R₁₁ is selected from the group consisting of hydrogen, C₁-C₆ alkyl, C₂-C₆ alkenyl, C₂-C₆ alkynyl, C₃-C₆ cycloalkyl, C₄-C₇ alkylcycloalkyl, C₁-C₇ alkylcarbonyl, C₁-C₇ aminocarbonyl, and C₂-C₅ alkoxycarbonyl;
R₁₂ is selected from the group consisting of hydrogen, C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl, C₃-C₆ cycloalkyl, —C(O)—C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl, 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, and C₃-C₆ cycloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s); and 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, —C(O)NH—C₃-C₆ cycloalkyl, and —C(O)NH—C₁-C₆ alkyl, wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl;
or
R₁₀ and W are taken together with the nitrogen to which they are attached to form a 4- to 7-membered ring optionally containing 1 to 2 heteroatoms selected from the group consisting of N, S, and O, wherein the carbons of the ring are optionally substituted with 1 to 4 substituents independently selected of cyano, hydroxyl, oxo, halogen, C₁-C₂ alkoxy, N,N-di-C₁-C₄-alkylaminocarboxyl, N—C₁-C₄-alkylaminocarboxyl, C₁-C₇ aminocarboxyl, C₁-C₄ alkyl optionally substituted with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, C₃-C₆ cycloalkyl optionally substituted with 1 to 3 substituents selected from the group consisting of halogen, cyano, hydroxyl, and C₁-C₄ alkoxy, —C(O)NH—C₃-C₆ cycloalkyl, —C(O)NH—C₁-C₆ alkyl, —C(O)NH—C₁-C₆ haloalkyl, 5- to 6-membered heteroaryl, and phenyl optionally substituted with 1 to 3 substituents independently selected from the group consisting of halogen, C₁-C₄ alkyl, cyano, hydroxyl, C₁-C₂ alkoxy, N,N-di-C₁-C₄-alkylaminocarboxyl, N—C₁-C₄-alkylaminocarboxyl, and C₁-C₇ aminocarboxyl, wherein any N in the 4- to 7-membered ring is substituted with a substituent selected from the group consisting of hydrogen, C₁-C₄ alkyl optionally substituted with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, C₃-C₆ cycloalkyl optionally substituted with 1 to 3 substituents independently selected from the group consisting of halogen, cyano, hydroxyl, C₁-C₄ alkoxy, —C(O)NH—C₃-C₆ cycloalkyl, and —C(O)NH—C₁-C₆ alkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered ring is optionally substituted with 1 or 2 oxygen atom(s); and
Y is C₁-C₆ alkyl optionally substituted with 1 to 5 substituents independently selected from the group consisting of halogen, cyano, hydroxyl, oxo, C₃-C₆ cycloalkyl, C₁-C₄ alkoxy, acetylenyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —SO₂NH(C₁-C₄ alkyl), —SO₂N(C₁-C₄ alkyl)₂, —SO₂NH(C₁-C₄ haloalkyl), —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, and —NH₂, —C(O)NH—C₁-C₆ alkyl, —C(O)NH—C₁-C₆ cyanoalkyl optionally substituted with 1 to 3 halogen, —C(O)NH—C₁-C₆ haloalkyl, phenyl optionally substituted with 1, 2 or 3 substituents independently selected from the group consisting of halogen, cyano, nitro, hydroxyl, C₁-C₄ alkyl optionally substituted with 1 to 3 substituents independently selected from the group consisting of halogen, cyano, hydroxyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl, and C₃-C₆ cycloalkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, C₂-C₆ alkenyl, and C₂-C₆ alkynyl, 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, C₁-C₇ aminocarbonyl, —NH(C₁-C₄ alkyl), —N(C₁-C₄ alkyl)₂, —C(O)NH—C₃-C₆ cycloalkyl, and —C(O)NH—C₁-C₆ alkyl, wherein any N in the heteroaryl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, and 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, and 5- to 6-membered heteroaryl, wherein any S in the 4- to 7-membered heterocycloalkyl or optionally benzo-fused 4- to 7-membered heterocycloalkyl is optionally substituted with 1 or 2 oxygen atom(s);
or a salt thereof.

In one embodiment, Z is CR₂.

In another embodiment, Z is N.

In one embodiment, the present invention also provides compositions, comprising: a compound of formula (I) or a salt thereof and at least one acceptable excipient, the composition optionally further comprising at least one additional active compound.

In one embodiment, the present invention also provides a method for treating pests, comprising: administering to a subject in need thereof an effective amount of a compound of formula (I) or a salt thereof, the method optionally further comprising an effective amount of at least one additional active compound.

In one embodiment, the present invention also provides a method for controlling pests, comprising: administering to a subject in need thereof an effective amount of a compound of formula (I) or a salt thereof, the method optionally further comprising an effective amount of at least one additional active compound.

In one embodiment, the present invention also provides a method for treating or controlling pests, comprising: contacting a subject's environment with an effective amount of a compound of formula (I) or a salt thereof, the method optionally further comprising an effective amount of at least one additional active compound.

Thus, the invention provides for the use of the compounds of the invention as a medicament, including for the manufacture of a medicament. In one embodiment, the invention provides the manufacture of a medicament comprising a compound of formula (I) or a salt thereof for treating parasites. In one embodiment, the invention provides the manufacture of a medicament comprising a compound of formula (I) or a salt thereof for controlling pests, preferably fleas or ticks. In another embodiment, the invention provides for the use of a compound of formula (I) or a salt thereof in the manufacture of a medicament for treating or controlling pests, preferably fleas or ticks.

The present invention also provides processes from making compounds of the invention and intermediates thereof.

### BRIEF DESCRIPTION OF THE DRAWINGS

FIG. 1 depicts an alternate synthesis scheme for preparing the compound of Examples 1.1 and 1.2, described herein.

FIG. 2 depicts an alternate synthesis scheme for preparing the compound of Examples 3.1 and 3.2, described herein.

### DETAILED DESCRIPTION

The term “C₁-C₂ alkyl” refers to an alkyl chain having from one to two carbon atoms and includes methyl and ethyl.

The term “C₁-C₄ alkyl” refers to a straight or branched alkyl chain having from one to four carbon atoms and includes methyl, ethyl, propyl, isopropyl, butyl, and the like.

Likewise, the term “C₁-C₆ alkyl” refers to a straight or branched alkyl chain having from one to six carbon atoms and includes methyl, ethyl, propyl, isopropyl, butyl, pentyl, hexyl and the like.

The terms “C₁-C₄ haloalkyl” and “C₁-C₄ halogenoalkyl” refers to a straight or branched alkyl chain having from one to four carbon atoms and 1 to 5 halogen and includes fluoromethyl, difluoromethyl, trifluoromethyl, 2,2,2-trifluoroethyl, 1,2,2-trifluoroethyl, 3,3,3-trifluoropropyl, and the like.

The terms “C₁-C₆ haloalkyl” and “C₁-C₆ halogenoalkyl” refers to a straight or branched alkyl chain having from one to six carbon atoms and 1 to 5 halogen and includes fluoromethyl, difluoromethyl, trifluoromethyl, 2,2,2-trifluoroethyl, 1,2,2-trifluoroethyl, 3,3,3-trifluoropropyl, 4,4,4-trifluorobutyl, and the like.

The term “C₂-C₆ alkenyl” refers to a straight or branched alkenyl chain having from two to four carbon atoms and one carbon-carbon double bond, and includes ethylene, propylene, iso-propylene, butylene, iso-butylene, sec-butylene, and the like.

The term “C₂-C₆ alkynyl” refers to a straight or branched alkynyl chain having from two to four carbon atoms and one carbon-carbon triple bond, and includes acetylene, propargyl, and the like.

The term “C₁-C₂ alkoxy” refers to a C₁-C₂ alkyl attached through an oxygen atom and includes methoxy and ethoxy.

The term “C₁-C₄ alkoxy” refers to a C₁-C₄ alkyl attached through an oxygen atom and includes methoxy, ethoxy, propoxy, isopropoxy, butoxy, and the like.

The term “C₁-C₆ alkoxy” refers to a C₁-C₆ alkyl attached through an oxygen atom and includes methoxy, ethoxy, propoxy, isopropoxy, butoxy, and the like.

The term “C₃-C₆ cycloalkyl” refers to an alkyl ring(s) of three to six carbon atoms, and includes cyclopropyl, cyclobutyl, cyclopentyl, and cyclohexyl. It is understood that the cycloalkyl rings can be fused, bridged, or spiro-fused.

The term “C₄-C₇ alkylcycloalkyl” refers to a C₁-C₄ alkyl substituted with a C₃-C₆ cycloalkyl such that the total number of carbons is four to seven and includes cyclopropylmethyl, cyclobutylmethyl, cyclopentylmethyl, cyclohexylmethyl, cyclopropylethyl, and the like.

The terms “halo” “halogen” and “halo” refers to chloro, fluoro, bromo or iodo atom(s).

The terms “4- to 7-membered heterocycloalkyl having 1 or 2 heteroatoms selected from the group O, S, N, wherein the heterocycloalkyl is optionally benzo-fused” and “4- to 7-membered heterocycloalkyl having 1 or 2 heteroatoms selected from the group O, S, B, N, wherein the heterocycloalkyl is optionally benzo-fused” refers to a 4- to 7-membered saturated or partially (but not fully) unsaturated ring having one or two heteroatoms selected from the group consisting of nitrogen, oxygen, and sulfur or having one or two heteroatoms selected from the group consisting of nitrogen, oxygen, boron, and sulfur and the ring optionally includes a carbonyl to form a lactam or lactone. It is understood that where sulfur is included that the sulfur may be either —S—, —SO—, or —SO₂—. The heterocyclic ring can be monocyclic or bicyclic and any bicyclic rings can be fused, bridged, or spiro-fused. The defined 4 to 7 members are exclusive of any optional benzo fused ring. Also, as will be fully appreciated by the skilled person, the saturated or partially (but not fully) unsaturated 4- to 7-membered heterocycloalkyl ring applies to the heterocycloalkyl ring and does not apply to any benzo fused ring, which by its nature will be fully unsaturated. It is further understood that the group can be attached as a substituent by any of the ring heteroatoms, valency permitting, the carbon atoms of the heterocycloalkyl, or the carbon atoms of any benzo-fused ring. It is also understood that when an optionally benzo-fused 4- to 7-membered heterocycloalkyl is optionally substituted on carbon, the substituents can be on the carbon atoms of the heterocycle and/or the benzo fused ring. For example, but not limiting, the term includes azetidinyl, pyrrolidinyl, piperidinyl, piperazinyl, morpholinyl, thiomorpholinyl, oxetanyl, thioxetanyl, dioxolanyl, tetrahydropyranyl, tetrahydrothiopyranyl, tetrahydrofuryl, hexahydropyrimidinyl, tetrahydropyrimidinyl, 2,6-diazaspiro[3.3]heptanyl, isoxazolidine, dihydroimidazolyl, indolyl, isoindolyl, and the like.

The term “5- or 6-membered heteroaryl” refers to a six membered, monocyclic, fully unsaturated ring with one to five carbon atoms and one or more, typically one to four, heteroatoms selected from the group consisting of nitrogen, oxygen, and sulfur. For example, but not limiting, the term includes pyrrolyl, furyl, thienyl, imidazolyl, oxazoyl, isoxazoyl, thiazolyl, triazolyl, pyrazinyl, pyrazolyl, pyridazinyl, pyridyl, pyrimidyl, and the like. It is understood that a 6-membered heteroaryl can be attached as a substituent through a ring carbon or a ring nitrogen atom where such an attachment mode is available.

Where R₁₀ and W are taken together with the nitrogen to which they are attached, the term “4- to 7-membered ring optionally containing 1 to 2 heteroatoms selected from the group consisting of N, S, and O” refers to a fully saturated or partially unsaturated (but not fully) ring having four to seven members inclusive of the nitrogen to which R₁₀ and W are attached and includes azetidinyl, pyrrolidinyl, piperidinyl, piperazinyl, morpholinyl, thiomorpholinyl, oxetanyl, dioxolanyl, tetrahydropyranyl, tetrahydrothiopyranyl, tetrahydrofuryl, hexahydropyrimidinyl, tetrahydropyrimidinyl, dihydroimidazolyl, and the like.

The term “5- to 10-membered heteroaryl having 1 or 2 heteroatoms selected from the group O, S, and N” refers to a five to ten membered, monocyclic or polycyclic fully unsaturated, ring or ring system with one to nine carbon atoms and one or two heteroatoms selected from the group consisting of nitrogen, oxygen, and sulfur. For example, but not limiting, the term includes furyl, thienyl, pyrrolyl, imidazolyl, isothiazolyl, isoxazolyl, oxadiazolyl, oxazolyl, thiazolyl, pyrazinyl, pyrazolyl, pyridazinyl, pyridyl, pyrimidyl, azepinyl, diazepinyl, benzofuryl, benzothienyl, indolyl, isoindolyl, benzimidazolyl, benzisothiazolyl, benzisoxazolyl, benzoxazolyl, benzopyrazinyl, benzopyrazolyl, quinazolyl, thienopyridyl, quinolyl, isoquinolyl benzothiazolyl, and the like. It is understood that a 5- to 10-membered heteroaryl having 1 or 2 heteroatoms selected from the group O, S, and N can be attached as a substituent through a ring carbon or a ring nitrogen atom where such an attachment mode is available.

The term “oxo” refers to an oxygen atom doubly bonded to the carbon to which it is attached to form the carbonyl of an amide, ketone, or aldehyde. For example, a pryidone radical is contemplated as an oxo substituted 6-membered heteroaryl.

The term “carboxyl” refers to the group below:

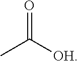

The term “N,N-di-C₁-C₄-alkylaminocarboxyl” refers to the group immediately below:

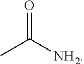

wherein the hydrogens on the nitrogen are substituted with two independently selected C₁-C₄ alkyl groups.

Likewise, term “N—C₁-C₄-alkylaminocarboxyl” refers to the group immediately below:

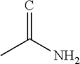

wherein one of the hydrogen on the nitrogen is substituted with a C₁-C₄ alkyl group.

The term “C₂-C₅ alkoxycarbonyl” refers the group below:

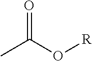

wherein R is a C₁-C₄ alkyl.

The term “C₂-C₇ alkylcarbonyl” refers the group below:

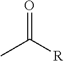

wherein R is a C₁-C₆ alkyl.

Likewise, the term “C₂-C₇ haloalkylcarbonyl” refers to the group immediately above wherein R is an C₁-C₆ haloalkyl.

The term “C₁-C₇ aminocarbonyl” refers to the group below:

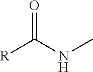

wherein R is a hydrogen or C₁-C₄ alkyl.

The term “nil” as used herein with reference to a group, substituent, moiety, or the like, indicates that that group, substituent, or moiety is not present. Wherein a group, substituent, or moiety is ordinarily bonded to two or more other groups, substituents, or moieties, the others are bonded together in lieu of the group, substituent, or moiety which is nil. For example, with a compound having the structure A-B-C; wherein B is nil, then A is directly bonded to C and the compound is A-C. As another example, with a compound having the structure A-B-C; wherein C is nil, then the compound is A-B.

The terms “salt” and “salts” refers to salts of veterinary or pharmaceutically acceptable organic acids and bases or inorganic acids and bases. Such salts are well known in the art and include those described in Journal of Pharmaceutical Science, 66, 2-19 (1977). An example is the hydrochloride salt.

The term “substituted,” including when used in “optionally substituted” refers to one or more hydrogen radicals of a group being replaced with non-hydrogen radicals (substituent(s)). It is understood that the substituents may be either the same or different at every substituted position. Combinations of groups and substituents envisioned by this invention are those that are stable or chemically feasible. For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “stable” refers to compounds that are not substantially altered when subjected to conditions to allow for their production. In a non-limiting example, a stable compound or chemically feasible compound is one that is not substantially altered when kept at a temperature of 40° C. or less, in the absence of moisture or other chemically reactive conditions, for about a week.

It is understood that, where the terms defined herein mention a number of carbon atoms, that the mentioned number refers to the mentioned group and does not include any carbons that may be present in any optional substituent(s) thereon or any carbons that may be present as part of a fused ring, including a benzo-fused ring.

The skilled artisan will appreciate that certain of the compounds of the present invention exist as isomers. All stereoisomers of the compounds of the invention, including geometric isomers, enantiomers, and diastereomers, in any ratio, are contemplated to be within the scope of the present invention.

As used herein, the term “(RS)” within chemical nomenclature refers to a racemic mixture at the indicated stereocenter.

As used herein, the term “(R or S)” or “(S or R)”, within chemical nomenclature refers to one of two possible configurations at the indicated stereocenter.

The skilled artisan will also appreciate that certain of the compounds of the present invention exist as tautomers. All tautomeric forms the compounds of the invention are contemplated to be within the scope of the present invention.

Compounds of the invention also include all isotopic variations, in which at least one atom of the predominant atom mass is replaced by an atom having the same atomic number, but an atomic mass different from the predominant atomic mass. Use of isotopic variations (e.g., deuterium, ²H) may afford greater metabolic stability. Additionally, certain isotopic variations of the compounds of the invention may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies.

The terms “compounds of the invention” and “a compound of the invention” and “compounds of the present invention” and the like include the embodiment of formula (I) and the other more particular embodiments encompassed by formula (I) described herein and the exemplified compounds described herein and a salt of each of these embodiments.

In an embodiment of a compound of formula (I),
R₁ may be hydrogen only when R₂ is trifluoromethyl, difluoromethyl, fluoro, chloro, or bromo;
R₃ may be hydrogen only when one of R₂ or R₄ is either trifluoromethyl, difluoromethyl, fluoro, chloro, or bromo; and
at most three of R₁, R₂, R₃, and R₄ are hydrogen.

In an embodiment of a compound of formula (I) where Z is CR₂,
R₁ may be hydrogen only when R₂ is trifluoromethyl, difluoromethyl, fluoro, chloro, or bromo;
R₃ may be hydrogen only when one of R₂ or R₄ is either trifluoromethyl, difluoromethyl, fluoro, chloro, or bromo; and
at most three of R₁, R₂, R₃, and R₄ are hydrogen.

In an embodiment of a compound of formula (I),
R₁ may be hydrogen only when R₂ is trifluoromethyl, difluoromethyl, or bromo;
R₃ may be hydrogen only when one of R₂ or R₄ is either trifluoromethyl, difluoromethyl, or bromo; and
at most three of R₁, R₂, R₃, and R₄ are hydrogen.

In an embodiment of a compound of formula (I) where Z is CR₂,
R₁ may be hydrogen only when R₂ is trifluoromethyl, difluoromethyl, or bromo;
R₃ may be hydrogen only when one of R₂ or R₄ is either trifluoromethyl, difluoromethyl, or bromo; and
at most three of R₁, R₂, R₃, and R₄ are hydrogen.

The compound of formula (I) having various embodiments as follows: formula (I):

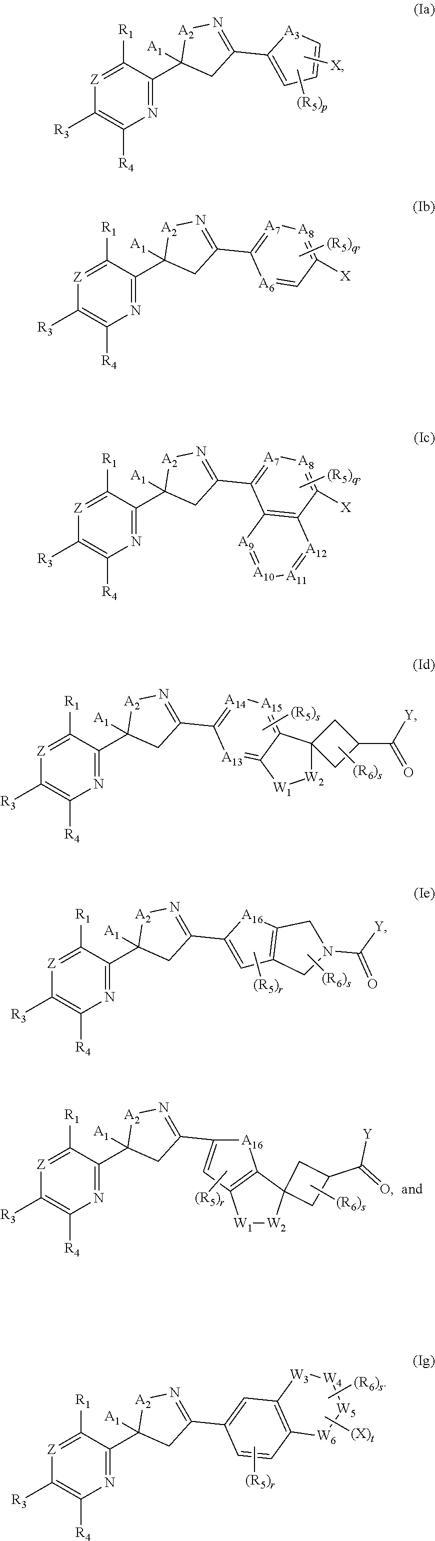

It is understood that for A₄, A₅, A₆, A₇, A₉, A₁₀, A₁₁, A₁₂, A₁₄, and/or A₁₅ an R₅ substituent, when present, takes the place of the hydrogen of the CH.

It is further understood that for the compound of formula (Ig) the X group, when present, is attached at W₃, W₄, W₅, or W₆ by replacing a hydrogen of a —CH₂— or —CH— group or the R of an —NR— group.

Further embodiments of compounds of the invention are provided below:
(1) One embodiment relates to a compound of formula (I) or a salt thereof.
(1.1) One embodiment relates to a compound of formula (I) or a salt thereof, where Z is CR₂.
(1.2) One embodiment relates to a compound of formula (I) or a salt thereof, where Z is N.
(a) One embodiment relates to compounds of formula (Ia) or a salt thereof.
(a1) One embodiment relates to compounds of formula (Ia) or a salt thereof, where Z is CR₂.
(a2) One embodiment relates to compounds of formula (Ia) or a salt thereof, where Z is N.
(b) One embodiment relates to compounds of formula (Ib) or a salt thereof.
(b1) One embodiment relates to compounds of formula (Ib) or a salt thereof, where Z is CR₂.
(b2) One embodiment relates to compounds of formula (Ib) or a salt thereof, where Z is N.
(c) One embodiment relates to compounds of formula (Ic) or a salt thereof.
(c1) One embodiment relates to compounds of formula (Ic) or a salt thereof, where Z is CR₂.
(c2) One embodiment relates to compounds of formula (Ic) or a salt thereof, where Z is N.
(d) One embodiment relates to compounds of formula (Id) or a salt thereof.
(d1) One embodiment relates to compounds of formula (Id) or a salt thereof, where Z is CR₂.
(d2) One embodiment relates to compounds of formula (Id) or a salt thereof, where Z is N.
(e) One embodiment relates to compounds of formula (Ie) or a salt thereof.
(e1) One embodiment relates to compounds of formula (Ie) or a salt thereof, where Z is CR₂.
(e2) One embodiment relates to compounds of formula (Ie) or a salt thereof, where Z is N.
(f) One embodiment relates to compounds of formula (If) or a salt thereof.
(f1) One embodiment relates to compounds of formula (If) or a salt thereof, where Z is CR₂.
(f2) One embodiment relates to compounds of formula (If) or a salt thereof, where Z is N.
(g) One embodiment relates to compounds of formula (Ig) or a salt thereof.
(g1) One embodiment relates to compounds of formula (Ig) or a salt thereof, where Z is CR₂.
(g2) One embodiment relates to compounds of formula (Ig) or a salt thereof, where Z is N.
(h) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethyl, R₃ is hydrogen, and R₄ is trifluoromethyl; or a salt thereof.
(i) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethyl, R₃ is hydrogen, and R₄ is bromo; or a salt thereof.
(j) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethyl, R₃ is hydrogen, and R₄ is fluoro; or a salt thereof.
(k) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethyl, R₃ is hydrogen, and R₄ is chloro; or a salt thereof.
(l) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethyl, R₃ is hydrogen, and R₄ is difluoromethyl; or a salt thereof.
(m) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethyl, R₃ is hydrogen, R₄ is methoxy; or a salt thereof.
(n) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, and R₄ is trifluoromethyl; or a salt thereof.
(o) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, and R₄ is bromo; or a salt thereof.
(p) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, and R₄ is fluoro; or a salt thereof.
(q) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, and R₄ is chloro; or a salt thereof.
(r) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, and R₄ is difluoromethyl; or a salt thereof.
(s) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, R₄ is methoxy; or a salt thereof.
(t) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is bromo, R₃ is hydrogen, and R₄ is trifluoromethyl; or a salt thereof.
(u) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is bromo, R₃ is hydrogen, and R₄ is bromo; or a salt thereof.
(v) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is bromo, R₃ is hydrogen, and R₄ is fluoro; or a salt thereof.
(w) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is bromo, R₃ is hydrogen, and R₄ is chloro; or a salt thereof.
(x) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g) (g1), and (g2) wherein R₁ is hydrogen, R₂ is chloro, R₃ is hydrogen, and R₄ is difluoromethyl; or a salt thereof.
(y) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is chloro, R₃ is hydrogen, R₄ is methoxy; or a salt thereof.
(z) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is fluoro, R₃ is hydrogen, and R₄ is trifluoromethyl; or a salt thereof.
(aa) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is fluoro, R₃ is hydrogen, and R₄ is bromo; or a salt thereof.
(ab) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is fluoro, R₃ is hydrogen, and R₄ is fluoro; or a salt thereof.
(ac) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is fluoro, R₃ is hydrogen, and R₄ is chloro; or a salt thereof.
(ad) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is fluoro, R₃ is hydrogen, and R₄ is difluoromethyl; or a salt thereof.
(ae) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is fluoro, R₃ is hydrogen, R₄ is methoxy; or a salt thereof.
(af) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), and (g2) wherein R₁ is hydrogen, R₂ is trifluoromethoxy, R₃ is hydrogen, R₄ is difluoromethyl; or a salt thereof.
(ag) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), (g2), (h), (i), (j), (k), (1), (m), (n), (o), (p), (q), (r), (s), (t), (u), (v), (w), (x), (y), (z), (aa), (ab), (ac), (ad), (ae), and (af) wherein A₂ is O; or a salt thereof.
(ah) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), (g2), (h), (i), (j), (k), (1), (m), (n), (o), (p), (q), (r), (s), (t), (u), (v), (w), (x), (y), (z), (aa), (ab), (ac), (ad), (ae), (af), and (ag) wherein A₁ is CF₃; or a salt thereof.
(ai) One embodiment relates to embodiments (1), (1.1), (1.2), (a), (a1), (a2), (b), (b1), (b2), (c), (c1), (c2), (d), (d1), (d2), (e), (e1), (e2), (f), (f1), (f2), (g), (g1), (g2), (h), (i), (j), (k), (1), (m), (n), (o), (p), (q), (r), (s), (t), (u), (v), (w), (x), (y), (z), (aa), (ab), (ac), (ad), (ae), (af), and (ag) wherein A₁ is CHF₂; or a salt thereof.
(aj) One embodiment relates to embodiments (1), (1.1), (1.2), (a), (a1), (a2), (h), (i), (j), (k), (1), (m), (n), (o), (p), (q), (r), (s), (t), (u), (v), (w), (x), (y), (z), (aa), (ab), (ac), (ad), (ae), (af), (ag), (ah), and (ai) wherein A₃ is S; or a salt thereof.
(ak) One embodiment relates to embodiments (1), (1.1), (1.2), (b), (b1), (b2), (h), (i), (j), (k), (1), (m), (n), (o), (p), (q), (r), (s), (t), (u), (v), (w), (x), (y), (z), (aa), (ab), (ac), (ad), (ae), (af), (ag), (ah), and (ai) wherein A₄, A₅, and A₆ are CH; or a salt thereof.
(al) One embodiment relates to embodiment (ak) wherein p is 1 and R₅ is C₁-C₆ alkyl; or a salt thereof.
(am) One embodiment relates to embodiment (a1) wherein R₅ is methyl; or a salt thereof.
(an) One embodiment relates to embodiments (1), (1.1), (1.2), (c), (c1), (c2), (h), (i), (j), (k), (1), (m), (n), (o), (p), (q), (r), (s), (t), (u), (v), (w), (x), (y), (z), (aa), (ab), (ac), (ad), (ae), (af), (ag), (ah), and (ai), wherein A₇, As, A₉, A₁₀, A₁₁, and A₁₂ are CH; or a salt thereof.
(ao) One embodiment relates to embodiments (1), (1.1), (1.2), (d), (d1), (d2), (h), (i), (j), (k), (1), (m), (n), (o), (p), (q), (r), (s), (t), (u), (v), (w), (x), (y), (z), (aa), (ab), (ac), (ad), (ae), (af), (ag), (ah), and (ai) wherein A₁₃, A₁₄, and A₁₅ are CH; or a salt thereof.
(ap) One embodiment relates to embodiment (ao) wherein W₁ is —CH₂— and W₂ is O; or a salt thereof.
(aq) One embodiment relates to embodiments (1), (1.1), (1.2), (a), (a1), (a2), (b), (b1), (b2), (c), (c1), (c2), (g), (g1), (g2), (h), (i), (j), (k), (1), (m), (n), (o), (p), (q), (r), (s), (t), (u), (v), (w), (x), (y), (z), (aa), (ab), (ac), (ad), (ae), (af), (ag), (ah), (ai), (aj), (ak), (a1), (am), (an), and (ao) wherein X is

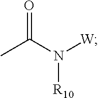

wherein R₁₀ is hydrogen; or a salt thereof.
(ar) One embodiment relates to embodiment (aq) wherein W is C₁-C₆ alkyl optionally substituted with 1 to 5 substituents independently selected from the group consisting of
   halogen,
   cyano,
   hydroxyl,
   oxo,
   C₁-C₄ alkoxy,
   C₃-C₆ cycloalkyl optionally substituted by 1 to 3 substituents independently selected from the group halogen and cyano,
   acetylenyl,
   —NH₂,
   C₁-C₇ aminocarbonyl,
   —NH(C₁-C₄ alkyl),
   —N(C₁-C₄ alkyl)₂,
   —SC₁-C₄ alkyl,
   —S(O)C₁-C₄ alkyl,
   —SO₂C₁-C₄ alkyl,
   —C(O)NH—C₃-C₆ cycloalkyl optionally substituted with 1 to 3 substituents independently selected from the group consisting of halogen,
      hydroxyl,
      cyano, and
      C₁-C₄ alkyl optionally substituted with 1 to 5 substituents independently
      selected from the group consisting of
         halogen,
         cyano,
         hydroxyl,
         C₁-C₄ alkoxy,
         C₃-C₆ cycloalkyl, and
         —NH₂,
   —C(O)NH—C₁-C₆ alkyl optionally substituted with 1 to 5 substituents independently selected from the group consisting of
      halogen,
      cyano,
      hydroxyl,
      C₁-C₄ alkoxy,
      C₃-C₆ cycloalkyl, and
      —NH₂;
   —C(O)NH—C₁-C₆ cyanoalkyl optionally substituted with 1 to 3 halogen,
   —C(O)NH—C₁-C₆ haloalkyl,
   —C(O)-4- to 7-membered heterocycloalkyl attached by a nitrogen and optionally having 1 or 2 other heteroatoms selected from the group O, S, N, wherein the carbons of the 4- to 7-membered heterocycloalkyl are optionally substituted with 1 to 4 substituents independently selected from the group consisting of
      halogen,
      cyano,
      nitro,
      hydroxyl,
      oxo,
      —NH₂,
      C₁-C₇ aminocarbonyl,
      C₁-C₄ alkyl optionally substituted with 1 to 5 substituents independently selected from the group consisting of
         halogen,
         cyano,
         hydroxyl,
         acetylenyl,
         oxo,
         C₁-C₄ alkoxy,
         —NH₂,
         C₁-C₇ aminocarbonyl,
         —NH(C₁-C₄ alkyl),
         N(C₁-C₄ alkyl)₂,
         SC₁-C₄ alkyl,
         —S(O)C₁-C₄ alkyl,
         SO₂C₁-C₄ alkyl,
         —C(O)NH—C₃-C₆ cycloalkyl, and
         —C(O)NH—C₁-C₆ alkyl, and
      C₃-C₆ cycloalkyl;
      and any other N in the 4- to 7-membered heterocycloalkyl, valency permitting, is substituted with a substituent selected from the group consisting of
         hydrogen,
         —NH₂,
         C₁-C₇ aminocarbonyl,
         —SO₂C₁-C₄ alkyl,
         —SO₂C₁-C₄ haloalkyl,
         C₁-C₄ alkyl optionally substituted with 1 to 5 substituents independently selected from the group consisting of
         halogen,
         cyano,
         hydroxyl,
         acetylenyl,
         C₁-C₄ alkoxy,
         —NH(C₁-C₄ alkyl),
         —N(C₁-C₄ alkyl)₂,
         —SC₁-C₄ alkyl,
         —S(O)C₁-C₄ alkyl,
         —SO₂C₁-C₄ alkyl,
         —C(O)NH—C₃-C₆ cycloalkyl,
         —C(O)NH—C₁-C₆ alkyl,
         —C(O)NH—C₁-C₆ haloalkyl;
   5- to 10-membered heteroaryl having 1 or 2 heteroatoms selected from the group O, S, and N and wherein the carbons of the 5- to 10-membered heteroaryl are optionally substituted with 1, 2 or 3 substituents independently selected from the group consisting of
      halogen,
      cyano,
      nitro,
      hydroxyl,
      C₁-C₄ alkyl optionally substituted with 1 to 5 substituents independently selected from the group consisting of
         halogen,
         cyano,
         hydroxyl,
         oxo,
         C₁-C₄ alkoxy,
         —NH₂,
         C₁-C₇ aminocarbonyl,
         —NH(C₁-C₄ alkyl),
         —N(C₁-C₄ alkyl)₂,
         —SC₁-C₄ alkyl,
         —S(O)C₁-C₄ alkyl,
         —SO₂C₁-C₄ alkyl,
         —C(O)NH—C₃-C₆ cycloalkyl,
         —C(O)NH—C₁-C₆ alkyl, and
         —C(O)NH—C₁-C₆ haloalkyl;
      C₃-C₆ cycloalkyl,
      C₁-C₄ haloalkyl,
      C₁-C₄ alkoxy,
      —NH₂,
      C₁-C₇ aminocarbonyl,
      —NH(C₁-C₄ alkyl),
      —N(C₁-C₄ alkyl)₂, and
      —C(O)NH—C₃-C₆ cycloalkyl;
   and any N in the heteroaryl, valency permitting, is optionally substituted with a substituent selected from the group consisting of
      hydrogen,
      C₁-C₄ alkyl optionally substituted with 1 to 5 substituents independently selected from the group consisting of
         halogen,
         cyano,
         hydroxyl,
         acetylenyl,
         oxo,
         C₃-C₆ cycloalkyl,
         C₁-C₄ alkoxy,
         —NH₂,
         C₁-C₇ aminocarbonyl,
         —NH(C₁-C₄ alkyl),
         —N(C₁-C₄ alkyl)₂,
         —SC₁-C₄ alkyl,
         —S(O)C₁-C₄ alkyl,
         —SO₂C₁-C₄ alkyl,
         —C(O)NH—C₃-C₆ cycloalkyl, and
         —C(O)NH—C₁-C₆ alkyl; and
      C₃-C₆ cycloalkyl;
   and any S in the heteroaryl is substituted with 1 or 2 oxygen atom(s);
   phenyl optionally substituted with 1 to 3 substituents selected from the group consisting of
      halogen,
      C₁-C₄ alkyl,
      cyano, or
      hydroxyl;
   C₃-C₆ cycloalkyl optionally substituted with 1 to 5 substituents independently selected from the group consisting of
      halogen,
      cyano,
      hydroxyl,
      oxo,
      C₁-C₄ alkoxy,
      C₁-C₄ alkyl optionally substituted with 1 to 3 groups selected from the
      group consisting of halogen and cyano,
      C₁-C₄ haloalkyl,
      —NH₂,
      C₁-C₇ aminocarbonyl,
      —NH(C₁-C₄ alkyl),
      —N(C₁-C₄ alkyl)₂,
      —SC₁-C₄ alkyl,
      —S(O)C₁-C₄ alkyl,
      —SO₂C₁-C₄ alkyl,
      —C(O)NH—C₃-C₆ cycloalkyl,
      —C(O)NH—C₁-C₆ alkyl,
      —C(O)NH—C₁-C₆ haloalkyl,
      C₂-C₆ alkenyl, and
      C₂-C₆ alkynyl; and
   4- to 7-membered heterocycloalkyl having 1 or 2 heteroatoms selected from the group O, S, B, N, wherein the heterocycloalkyl is optionally benzo-fused, and wherein the carbons of the 4- to 7-membered heterocycloalkyl or optionally benzo-fused 4- to 7-membered heterocycloalkyl are optionally substituted with 1 to 4 substituents independently selected from the group consisting of
      halogen,
      cyano,
      nitro,
      hydroxyl,
      oxo,
      C₁-C₄ alkyl optionally substituted with 1 to 5 substituents independently selected from the group consisting of
         halogen,
         cyano, hydroxyl,
         acetylenyl,
         oxo,
         C₁-C₄ alkoxy,
         C₃-C₆ cycloalkyl,
         —NH₂,
         C₁-C₇ aminocarbonyl,
         —NH(C₁-C₄ alkyl),
         —N(C₁-C₄ alkyl)₂,
         —SC₁-C₄ alkyl,
         —S(O)C₁-C₄ alkyl,
         —SO₂C₁-C₄ alkyl,
         —C(O)NH—C₃-C₆ cycloalkyl,
         —C(O)NH—C₁-C₆ alkyl, and
         and —C(O)NH—C₁-C₆ haloalkyl;
   and any B in the of the 4- to 7-membered heterocycloalkyl or optionally benzo-fused 4- to 7-membered heterocycloalkyl, valency permitting, is substituted with hydroxyl,
   and any N in the 4- to 7-membered heterocycloalkyl or optionally benzo-fused 4- to 7-membered heterocycloalkyl, valency permitting, is substituted with a substituent selected from the group consisting of
      hydrogen,
      —NH₂,
      C₁-C₇ aminocarbonyl,
      —SO₂C₁-C₄ alkyl,
      —SO₂C₁-C₄ haloalkyl,
      —C(O)—NH₂,
      C₁-C₄ alkyl optionally substituted with 1 to 5 substituents independently selected from the group consisting of
         halogen,
         cyano,
         hydroxyl,
         acetylenyl,
         C₁-C₄ alkoxy,
         —NH(C₁-C₄ alkyl),
         —N(C₁-C₄ alkyl)₂,
         —SC₁-C₄ alkyl,
         —S(O)C₁-C₄ alkyl,
         —SO₂C₁-C₄ alkyl,
         —C(O)NH—C₃-C₆ cycloalkyl, and
         —C(O)NH—C₁-C₆ haloalkyl;
      C₃-C₆ cycloalkyl;
      5- to 6-membered heteroaryl; and
      phenyl optionally substituted with 1 to 3 substituents independently selected from the group consisting of
         halogen,
         C₁-C₄ alkyl,
         cyano, and
         hydroxyl; and
   any S in the 4- to 7-membered heterocycloalkyl or optionally benzo-fused 4- to 7-membered heterocycloalkyl is substituted with 1 or 2 oxygen atom(s);
or a salt thereof.
(as) One embodiment related to embodiment (ar) wherein W is a C₁-C₆ alkyl substituted with a substituent selected from the group consisting of
—C(O)NH—C₁-C₆ alkyl optionally substituted with 1 to 5 substituents independently selected from the group consisting of
   halogen,
   cyano,
   hydroxyl,
   C₁-C₄ alkoxy,
   C₃-C₆ cycloalkyl, and
   —NH₂;
C(O)NH—C₁-C₆ cyanoalkyl optionally substituted with 1 to 3 halogen,
—C(O)NH—C₁-C₆ haloalkyl, and
—C(O)-4- to 7-membered heterocycloalkyl attached by a nitrogen and optionally having 1
or 2 other heteroatoms selected from the group O, S, N, wherein the carbons of the 4- to 7-membered heterocycloalkyl are optionally substituted with 1 to 4 substituents independently selected from the group consisting of
   halogen,
   cyano,
   nitro,
   hydroxyl,
   oxo,
   —NH₂,
   C₁-C₇ aminocarbonyl,
   C₁-C₄ alkyl optionally substituted with 1 to 5 substituents independently selected from the group consisting of
      halogen,
      cyano,
      hydroxyl,
      acetylenyl,
      oxo,
      C₁-C₄ alkoxy,
      —NH₂,
      C₁-C₇ aminocarbonyl,
      —NH(C₁-C₄ alkyl),
      N(C₁-C₄ alkyl)₂,
      SC₁-C₄ alkyl,
      —S(O)C₁-C₄ alkyl,
      SO₂C₁-C₄ alkyl,
      —C(O)NH—C₃-C₆ cycloalkyl, and
      —C(O)NH—C₁-C₆ alkyl, and
   C₃-C₆ cycloalkyl;
   and any other N in the 4- to 7-membered heterocycloalkyl, valency permitting, is substituted with a substituent selected from the group consisting of
      hydrogen,
      —NH₂,
      C₁-C₇ aminocarbonyl,
      —SO₂C₁-C₄ alkyl,
      —SO₂C₁-C₄ haloalkyl,
      C₁-C₄ alkyl optionally substituted with 1 to 5 substituents independently selected from the group consisting of
         halogen,
         cyano,
         hydroxyl,
         acetylenyl,
         C₁-C₄ alkoxy,
         —NH(C₁-C₄ alkyl),
         —N(C₁-C₄ alkyl)₂,
         —SC₁-C₄ alkyl,
         —S(O)C₁-C₄ alkyl,
         —SO₂C₁-C₄ alkyl,
         —C(O)NH—C₃-C₆ cycloalkyl,
         —C(O)NH—C₁-C₆ alkyl,
         —C(O)NH—C₁-C₆ haloalkyl;
or a salt thereof.
(at) One embodiment relates to embodiment (as) wherein W is

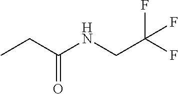

or a salt thereof.
(au) One embodiment relates to embodiment (aq) wherein W is C₁-C₆ alkyl substituted with —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, and —NH₂; or a salt thereof.
(av) One embodiment relates to embodiment (aq) wherein W is C₁-C₆ alkyl substituted with —C(O)NH—C₁-C₆ alkyl optionally substituted with 1 to 5 substituents independently selected from the group consisting of
   halogen,
   cyano,
   hydroxyl,
   C₁-C₄ alkoxy,
   C₃-C₆ cycloalkyl, and
   —NH₂;
or a salt thereof.
(av1) One embodiment relates to embodiment (av) wherein W is

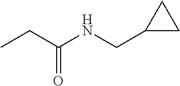

or a salt thereof.
(av2) One embodiment relates to embodiment (av) where in W is

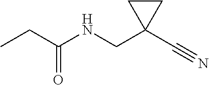

or a salt thereof.
(aw) One embodiment relates to embodiment (aq) wherein W is C₁-C₆ alkyl substituted with a 5- to 10-membered heteroaryl having 1 or 2 heteroatoms selected from the group O, S, B, and N and wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl; C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl; and any B in the heteroaryl is substituted with hydroxyl and any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl and any S in the heteroaryl is substituted with 1 or 2 oxygen atom(s); or a salt thereof.
(aw1) One embodiment relates to embodiment (aq) wherein W is C₁-C₆ alkyl substituted with a pyridine optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl; C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl; or a salt thereof.
(aw2) One embodiment relates to embodiment (aq) wherein W is C₁-C₆ alkyl substituted with a thiazole optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl; C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl; or a salt thereof.
(ax) One embodiment relates to embodiment (aq) wherein W is a 4- to 7-membered heterocycloalkyl having 1 or 2 heteroatoms selected from the group O, S, N, wherein the heterocycloalkyl is optionally benzo-fused, and wherein the carbons of the 4- to 7-membered heterocycloalkyl or optionally benzo-fused 4- to 7-membered heterocycloalkyl are optionally substituted with 1 to 4 substituents independently selected from the group consisting of
   halogen,
   cyano,
   nitro,
   hydroxyl,
   oxo,
   C₁-C₄ alkyl optionally substituted with 1 to 5 substituents independently selected from the group consisting of
      halogen,
      cyano, hydroxyl,
      acetylenyl,
      oxo,
      C₁-C₄ alkoxy,
      C₃-C₆ cycloalkyl,
      —NH₂,
      C₁-C₇ aminocarbonyl,
      —NH(C₁-C₄ alkyl),
      —N(C₁-C₄ alkyl)₂,
      —SC₁-C₄ alkyl,
      —S(O)C₁-C₄ alkyl,
      —SO₂C₁-C₄ alkyl,
      —C(O)NH—C₃-C₆ cycloalkyl,
      —C(O)NH—C₁-C₆ alkyl,
      and
      —C(O)NH—C₁-C₆ haloalkyl; and
and any N in the 4- to 7-membered heterocycloalkyl or optionally benzo-fused 4- to 7-membered heterocycloalkyl, valency permitting, is substituted with a substituent selected from the group consisting of
   hydrogen,
   —NH₂,
   C₁-C₇ aminocarbonyl,
   —SO₂C₁-C₄ alkyl,
   —SO₂C₁-C₄ haloalkyl,
   —C(O)—NH₂,
   C₁-C₄ alkyl optionally substituted with 1 to 5 substituents independently selected from the group consisting of
      halogen,
      cyano,
      hydroxyl,
      acetylenyl,
      C₁-C₄ alkoxy,
      C₃-C₆ cycloalkyl,
      —NH(C₁-C₄ alkyl),
      —N(C₁-C₄ alkyl)₂,
      —SC₁-C₄ alkyl,
      —S(O)C₁-C₄ alkyl,
      —SO₂C₁-C₄ alkyl,
      —C(O)NH—C₃-C₆ cycloalkyl, and
      —C(O)NH—C₁-C₆ haloalkyl;
   C₃-C₆ cycloalkyl;
   5- to 6-membered heteroaryl; and
   phenyl optionally substituted with 1 to 3 substituents independently selected from the group consisting of
      halogen,
      C₁-C₄ alkyl,
      cyano, and
      hydroxyl; and
any S in the 4- to 7-membered heterocycloalkyl or optionally benzo-fused 4- to 7-membered heterocycloalkyl is substituted with 1 or 2 oxygen atom(s);
or a salt thereof.
(ax1) One embodiment relates to embodiment (ax) wherein W is a 4- to 7-membered heterocycloalkyl having 1 or 2 heteroatoms selected from the group O, S, N is selected from the group consisting or pyrrolyl, azetidinyl, 2-oxoazetidinyl, isoxazolidinyl, 2,6-diazaspiro[3.3]heptanyl, and 1,6-diazaspiro[3.3]heptanyl wherein the carbons of the 4- to 7-membered heterocycloalkyl are optionally substituted with 1 to 4 substituents independently selected from the group consisting of
   halogen,
   cyano,
   nitro,
   hydroxyl,
   oxo,
   C₁-C₄ alkyl optionally substituted with 1 to 5 substituents independently selected from the group consisting of
      halogen,
      cyano, hydroxyl,
      acetylenyl,
      oxo,
      C₁-C₄ alkoxy,
      C₃-C₆ cycloalkyl,
      —NH₂,
      C₁-C₇ aminocarbonyl,
      —NH(C₁-C₄ alkyl),
      —N(C₁-C₄ alkyl)₂,
      —SC₁-C₄ alkyl,
      —S(O)C₁-C₄ alkyl,
      —SO₂C₁-C₄ alkyl,
      —C(O)NH—C₃-C₆ cycloalkyl,
      —C(O)NH—C₁-C₆ alkyl,
      and
      —C(O)NH—C₁-C₆ haloalkyl;
and any N in the 4- to 7-membered heterocycloalkyl 4- to 7-membered heterocycloalkyl, valency permitting, is substituted with a substituent selected from the group consisting of
   hydrogen,
   —SO₂C₁-C₄ alkyl,
   —SO₂C₁-C₄ haloalkyl,
   —C(O)—NH₂,
   C₁-C₄ alkyl optionally substituted with 1 to 5 substituents independently selected from the group consisting of
      halogen,
      cyano,
      hydroxyl,
      acetylenyl,
      C₁-C₄ alkoxy,
      C₃-C₆ cycloalkyl,
      —NH(C₁-C₄ alkyl),
      —N(C₁-C₄ alkyl)₂,
      —SC₁-C₄ alkyl,
      —S(O)C₁-C₄ alkyl,
      —SO₂C₁-C₄ alkyl,
      —C(O)NH—C₃-C₆ cycloalkyl, and
      —C(O)NH—C₁-C₆ haloalkyl; and
   C₃-C₆ cycloalkyl;
or a salt thereof.
(ay) One embodiment relates to embodiments (ax) and (ax1) wherein the carbons of the 4- to 7-membered heterocycloalkyl is optionally substituted with 1 to 2 substituents independently selected from the group consisting of oxo and C₁-C₄ alkyl and any N in the 4- to 7-membered heterocycloalkyl, valency permitting, is substituted by hydrogen and C₁-C₄ alkyl optionally substituted with 1 to 3 halogen, cyano, acetylenyl, or
C₃-C₆ cycloalkyl; or a salt thereof.
(az) One embodiment relates to embodiments (ax), (ax1), and (ay) wherein the carbons of the 4- to 7-membered heterocycloalkyl are substituted with 1 oxo and any N in the 4- to 7-membered heterocycloalkyl, valency permitting, is substituted by C₁-C₄ alkyl substituted by 1 cyano; or a salt thereof.
(ba) One embodiment relates to embodiments (ax), (ax1), and (ay) wherein the carbons of the 4- to 7-membered heterocycloalkyl are substituted with 1 oxo and any N in the 4- to 7-membered heterocycloalkyl, valency permitting, is substituted by C₁-C₄ alkyl substituted with 1 to 3 halogens; or a salt thereof.
(bb) One embodiment relates to embodiments (ax), (ax1), and (ay) wherein the carbons of the 4- to 7-membered heterocycloalkyl are substituted with 1 oxo and any N in the 4- to 7-membered heterocycloalkyl, valency permitting, is substituted by C₁-C₄ alkyl substituted with 1 C₃-C₆ cycloalkyl; or a salt thereof.
(bc) One embodiment relates to embodiments (ax), (ax1), and (ay) wherein any N in the 4- to 7-membered heterocycloalkyl, valency permitting, is substituted by C₁-C₄ alkyl substituted with 1 cyano; or a salt thereof.
(bd) One embodiment relates to embodiments (ax), (ax1), and (ay) wherein any N in the 4- to 7-membered heterocycloalkyl, valency permitting, is substituted by C₁-C₄ alkyl substituted with 1 to 3 halogens; or a salt thereof.
(be) One embodiment relates to embodiments (ax), (ax1), and (ay) wherein any N in the 4- to 7-membered heterocycloalkyl, valency permitting, is substituted by C₁-C₄ alkyl substituted with 1 C₃-C₆ cycloalkyl; or a salt thereof.
(be1) One embodiment relates to embodiment (aq) wherein W is a C₃-C₆ cycloalkyl optionally substituted with 1 to 5 substituents independently selected from the group consisting of
   halogen,
   cyano,
   hydroxyl,
   oxo,
   C₁-C₄ alkoxy,
   C₁-C₄ alkyl optionally substituted with 1 to 3 groups selected from the group consisting of halogen and cyano,
   C₁-C₄ haloalkyl,
   —NH₂,
   C₁-C₇ aminocarbonyl,
   —NH(C₁-C₄ alkyl),
   —N(C₁-C₄ alkyl)₂,
   —SC₁-C₄ alkyl,
   —S(O)C₁-C₄ alkyl,
   —SO₂C₁-C₄ alkyl,
   —C(O)NH—C₃-C₆ cycloalkyl,
   —C(O)NH—C₁-C₆ alkyl,
   —C(O)NH—C₁-C₆ haloalkyl,
   C₂-C₆ alkenyl optionally substituted with 1 to 3 halogens; and
   C₂-C₆ alkynyl;
or a salt thereof.
(bf) One embodiment relates to embodiments (1), (1.1), (1.2), (d), (d1), (d2), (e), (e1), (e2), (f), (f1), (f2), (h), (i), (j), (k), (1), (m), (n), (o), (p), (q), (r), (s), (t), (u), (v), (w), (x), (y), (z), (aa), (ab), (ac), (ad), (ae), (af), (ag), (ah), (aj), (ak), (ao), and (ap) wherein Y is C₁-C₆ alkyl optionally substituted with 1 to 5 substituents independently selected from the group consisting of
   halogen,
   cyano,
   hydroxyl,
   oxo,
   C₃-C₆ cycloalkyl,
   C₁-C₄ alkoxy,
   acetylenyl,
   —NH₂,
   C₁-C₇ aminocarbonyl,
   —NH(C₁-C₄ alkyl),
   —N(C₁-C₄ alkyl)₂,
   —SC₁-C₄ alkyl,
   —S(O)C₁-C₄ alkyl,
   —SO₂C₁-C₄ alkyl,
   —C(O)NH—C₃-C₆ cycloalkyl optionally substituted with 1 to 3 substituents independently selected from the group consisting of
      halogen,
      hydroxyl,
      cyano, and
      C₁-C₄ alkyl optionally substituted with 1 to 5 substituents independently selected from the group consisting of
         halogen,
         cyano,
         hydroxyl,
         C₁-C₄ alkoxy, and
         —NH₂,
   —C(O)NH—C₁-C₆ alkyl,
   —C(O)NH—C₁-C₆ cyanoalkyl optionally substituted with 1 to 3 halogen,
   —C(O)NH—C₁-C₆ haloalkyl;
or a salt thereof.
(bg) One embodiment relates to embodiment (bf) wherein Y is C₁-C₆ alkyl substituted with 1—SO₂C₁-C₄ alkyl; or a salt thereof.
(bh) One embodiment relates to embodiment (bf) wherein Y is C₁-C₆ alkyl substituted with 1 —SO₂CH₃; or a salt thereof.
(bi) One embodiment relates to embodiment (w) wherein W is

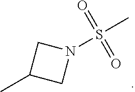

(xa) Another embodiment relates to each of the exemplified compounds or a salt thereof.
(xb) Another embodiment relates to each stereoisomer of each exemplified, depicted, or named compound; or a salt thereof.
(xc) Another embodiment relates to a salt of each of the exemplified compounds.

The compounds of the invention can be prepared by a variety of procedures many of which are already described in the art. For example, see WO 2005/085216, WO 2007/079162, US 2007/066617, US20130131017, WO 2009/002809, WO 2009/112275, WO 2010/003923, WO 2010/070068, WO 2012/120399, WO 2013/079407 and WO 2021/127188.

The following examples are intended to be illustrative and non-limiting, and represent specific embodiments of the present invention.

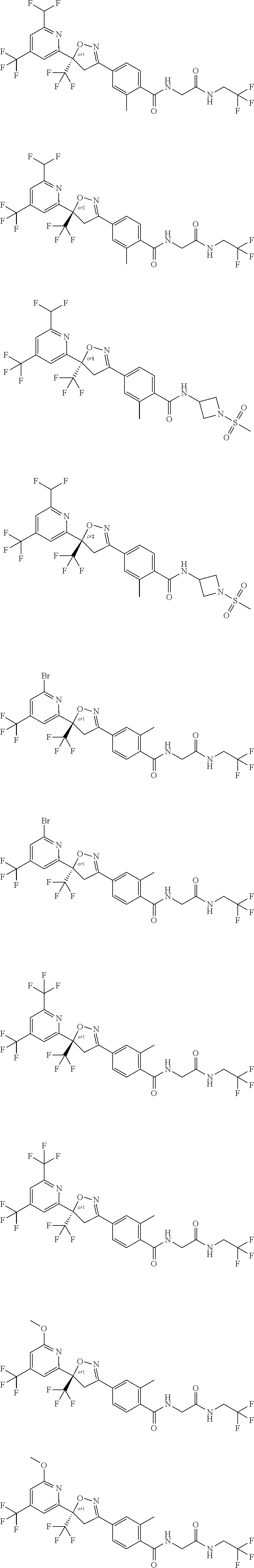

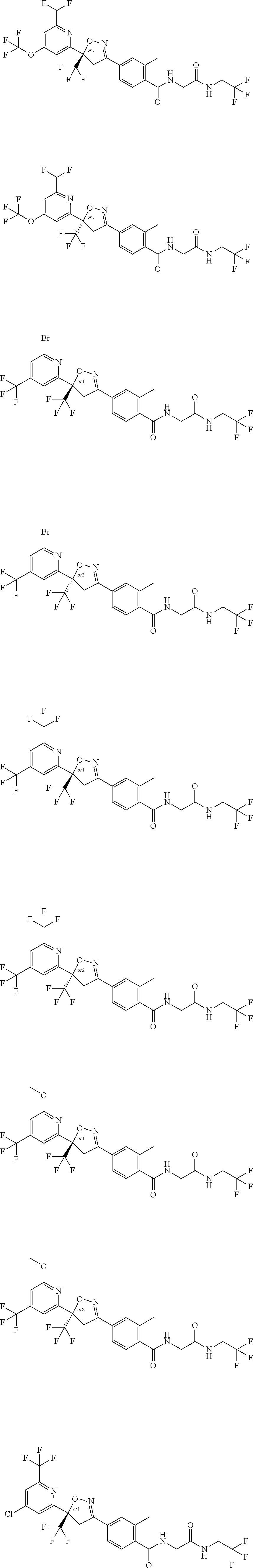

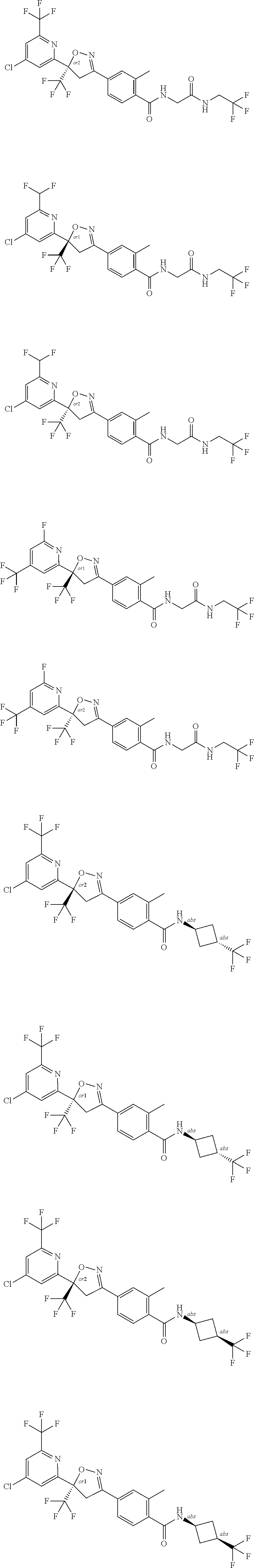

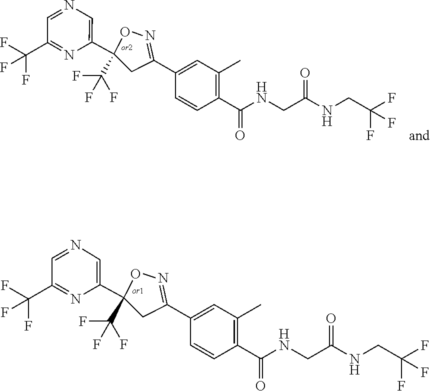 [SMILES: Cc1cc(C2=NO[C@@](c3cncc(C(F)(F)F)n3)(C(F)(F)F)C2)ccc1C(=O)NCC(=O)NCC(F)(F)F ; "and" ; Cc1cc(C2=NO[C@](c3cncc(C(F)(F)F)n3)(C(F)(F)F)C2)ccc1C(=O)NCC(=O)NCC(F)(F)F]

and each stereoisomer of the compounds above.)

In another aspect, disclosed are compounds of formula (II), or a salt thereof,

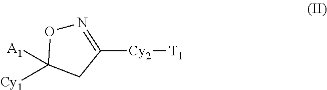

wherein,
A₁ is —CF₃, —CHF₂, —CH₂F, or —CF₂CF₃;
Cy₁ is selected from:

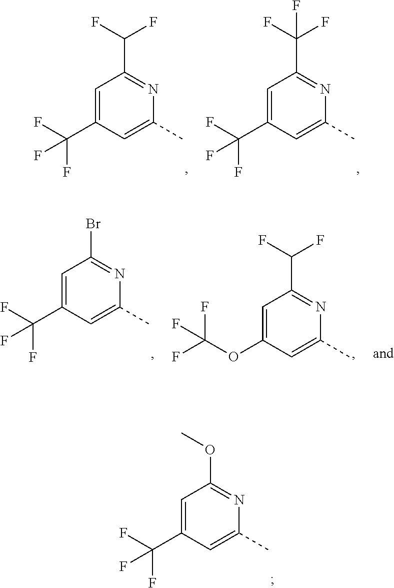

Cy₂ is selected from:

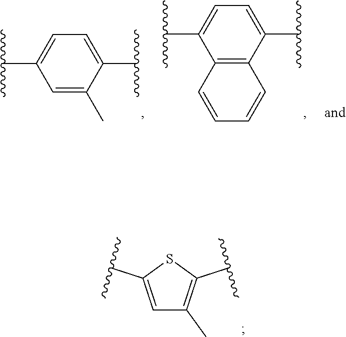

and

T₁ is selected from:

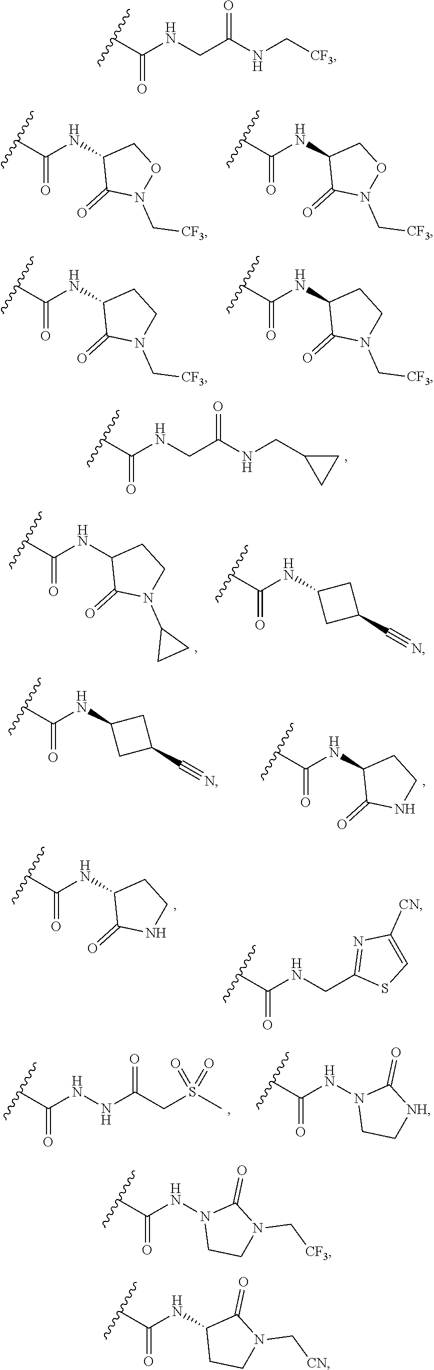

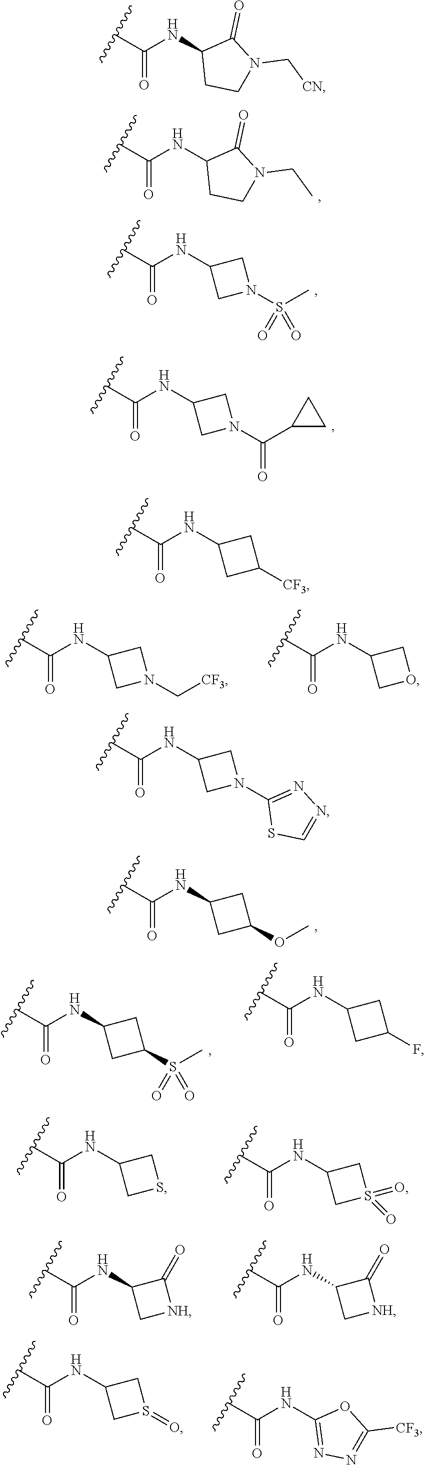

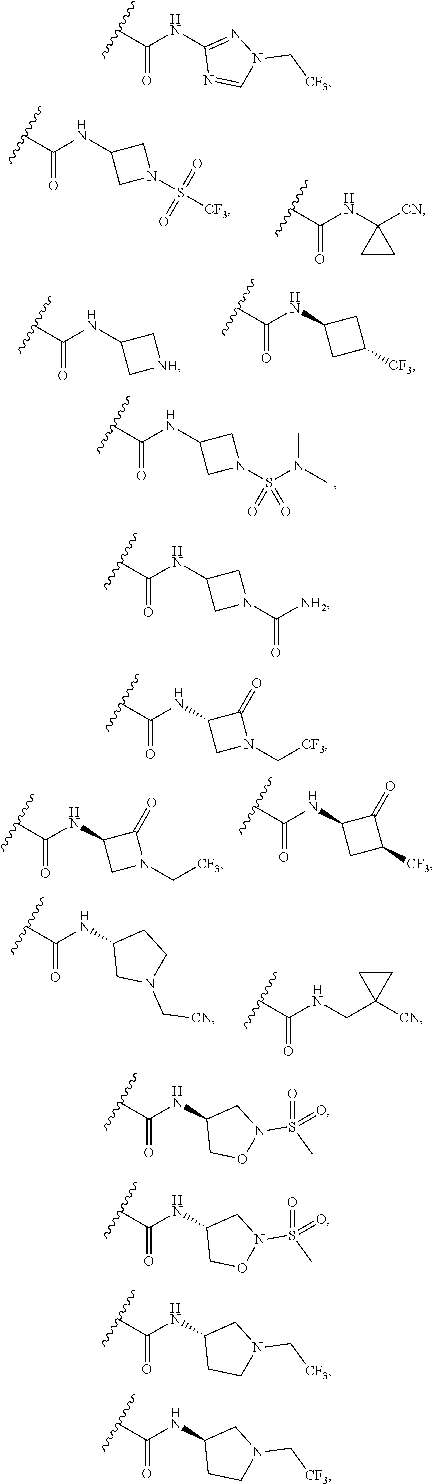

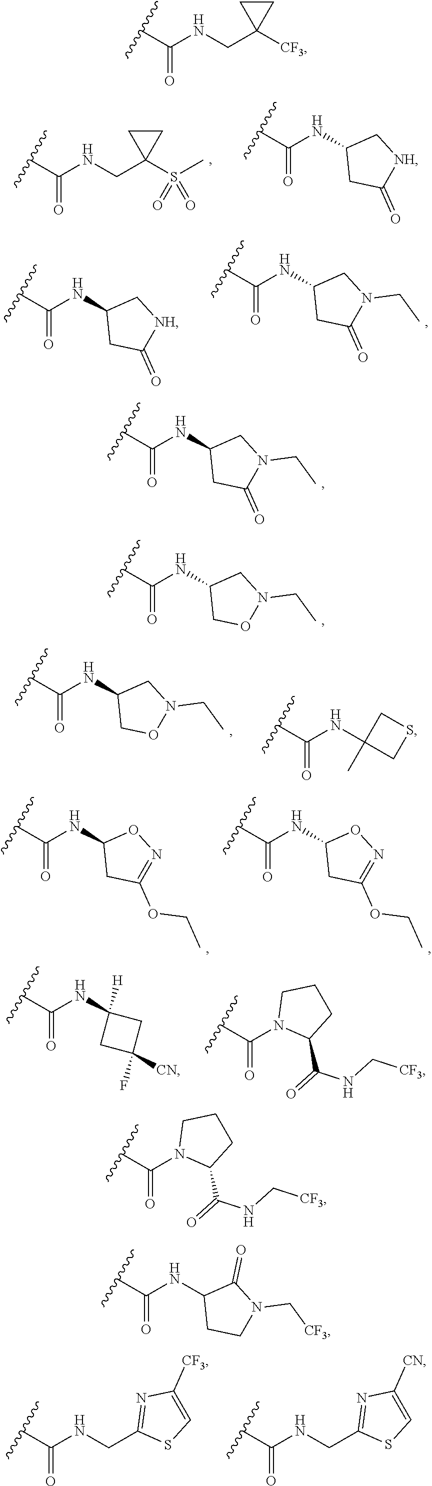

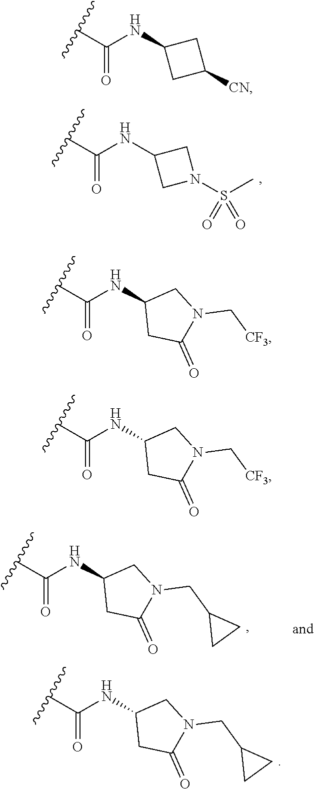 [SMILES: *C(=O)N[C@H]1C[C@@H](C#N)C1 ; *C(=O)NC1CN(S(C)(=O)=O)C1 ; *C(=O)N[C@@H]1CC(=O)N(CC(F)(F)F)C1 ; *C(=O)N[C@H]1CC(=O)N(CC(F)(F)F)C1 ; *C(=O)N[C@@H]1CC(=O)N(CC2CC2)C1 ; *C(=O)N[C@H]1CC(=O)N(CC2CC2)C1]

In an embodiment, disclosed are compounds of formula (IIa), or a salt thereof,

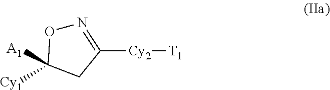

wherein A₁, Cy₁, Cy₂, and T₁ are as defined above.

In another embodiment, disclosed are compounds of formula (IIb), or a salt thereof,

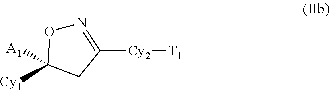

wherein A₁, Cy₁, Cy₂, and T₁ are as defined above.

Further embodiments of the invention are provided below:
(2) One embodiment relates to compounds of formula (II) or a salt thereof.
(2a) One embodiment relates to compounds of formula (IIa) or a salt thereof.
(2b) One embodiment relates to compounds of formula (IIb) or a salt thereof.
(2c) One embodiment relates to embodiments (2), (2a), and (2b) wherein A₁ is —CF₃.
(2d) One embodiment relates to embodiments (2), (2a), and (2b) wherein A₁ is —CHF₂.
(2e) One embodiment relates to embodiments (2), (2a), and (2b) wherein A₁ is —CH₂F.
(2f) One embodiment relates to embodiments (2), (2a), and (2b) wherein A₁ is —CF₂CF₃.
(2g) One embodiment relates to embodiments (2), (2a), (2b), (2c), (2d), (2e), and (2f)
wherein Cy₁ is

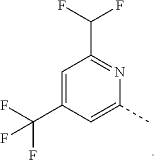

(2h) One embodiment relates to embodiments (2), (2a), (2b), (2c), (2d), (2e), and (2f)
wherein Cy₁ is

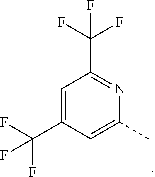

(2i) One embodiment relates to embodiments (2), (2a), (2b), (2c), (2d), (2e), and (2f)
wherein Cy₁ is

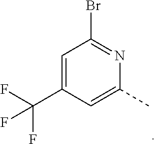

(2j) One embodiment relates to embodiments (2), (2a), (2b), (2c), (2d), (2e), and (2f) wherein Cy₁ is

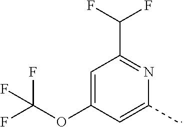

(2k) One embodiment relates to embodiments (2), (2a), (2b), (2c), (2d), (2e), and (2f) wherein Cy₁ is

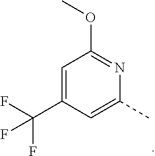

(2l) One embodiment relates to embodiments (2), (2a), (2b), (2c), (2d), (2e), (2f), (2g), (2h), (2i), (2j), and (2k) wherein Cy₂ is

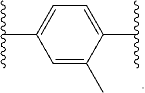 [SMILES: *c1ccc(*)c(C)c1]

(2m) One embodiment relates to embodiments (2), (2a), (2b), (2c), (2d), (2e), (2f), (2g), (2h), (2i), (2j), and (2k) wherein Cy₂ is

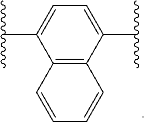 [SMILES: *c1ccc(*)c2ccccc12]

(2n) One embodiment relates to embodiments (2), (2a), (2b), (2c), (2d), (2e), (2f), (2g), (2h), (2i), (2j), and (2k) wherein Cy₂ is

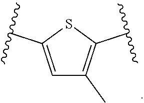 [SMILES: *c1cc(C)c(*)s1]

(2o) One embodiment relates to embodiments (2), (2a), (2b), (2c), (2d), (2e), (2f), (2g), (2h), (2i), (2j), (2k), (2l), (2m), and (2n) wherein T₁ is

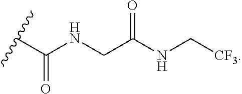 [SMILES: *C(=O)NCC(=O)NCC(F)(F)F]

In another aspect, disclosed are compounds of formula (III), or a salt thereof,

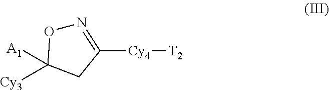

wherein,
A₁ is —CF₃ or —CHF₂;
Cy₃ is selected from:

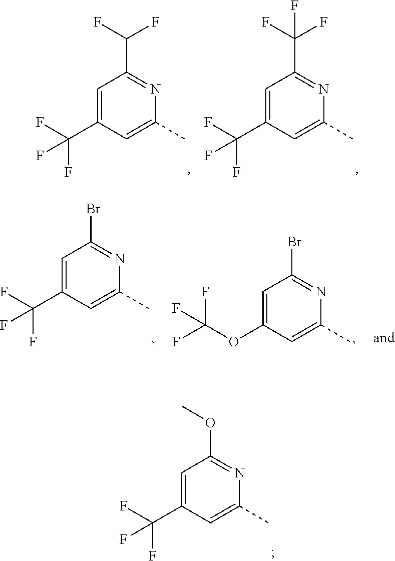

Cy₄ is:

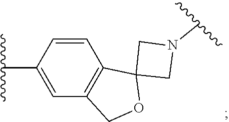 [SMILES: *c1ccc2c(c1)COC21CN(*)C1]

and
T₂ is selected from:

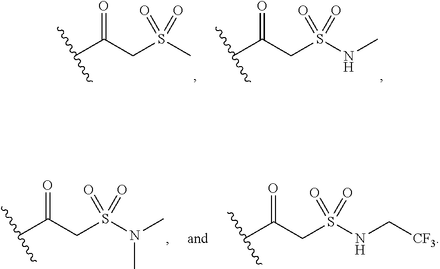

In an embodiment, disclosed are compounds of formula (IIIa), or a salt thereof,

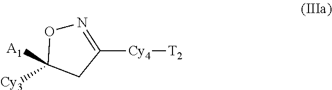

wherein A₁, Cy₃, Cy₄, and T₂ are as defined above.

In another embodiment, disclosed are compounds of formula (IIIb), or a salt thereof,

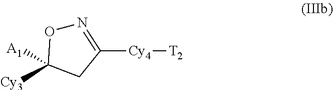

wherein A₁, Cy₃, Cy₄, and T₂ are as defined above.

Further embodiments of the invention are provided below:
(3) One embodiment relates to compounds of formula (III) or a salt thereof.
(3a) One embodiment relates to compounds of formula (IIa) or a salt thereof.
(3b) One embodiment relates to compounds of formula (IIb) or a salt thereof.
(3c) One embodiment relates to embodiments (3), (3a), and (3b) wherein A₁ is —CF₃.
(3d) One embodiment relates to embodiments (3), (3a), and (3b) wherein A₁ is —CHF₂.
(3e) One embodiment relates to embodiments (3), (3a), and (3b) wherein A₁ is —CH₂F.
(3f) One embodiment relates to embodiments (3), (3a), and (3b) wherein A₁ is —CF₂CF₃.
(3g) One embodiment relates to embodiments (3), (3a), (3b), (3c), (3d), (3e), and (3f)
wherein Cy₃ is

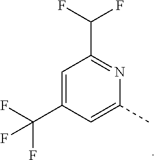

(3h) One embodiment relates to embodiments (3), (3a), (3b), (3c), (3d), (3e), and (3f)
wherein Cy₃ is

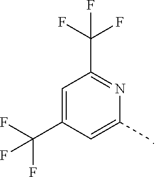

(3i) One embodiment relates to embodiments (3), (3a), (3b), (3c), (3d), (3e), and (3f) wherein Cy₃ is

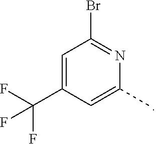

(3j) One embodiment relates to embodiments (3), (3a), (3b), (3c), (3d), (3e), and (3f) wherein Cy₃ is

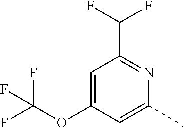

(3k) One embodiment relates to embodiments (3), (3a), (3b), (3c), (3d), (3e), and (3f) wherein Cy₃ is

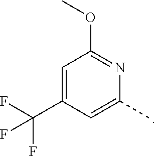

(3l) One embodiment relates to embodiments (3), (3a), (3b), (3c), (3d), (3e), (3f), (3g), (3h), (3i), (3j), and (3k) wherein Cy₄ is

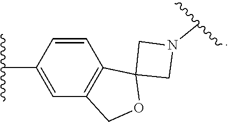 [SMILES: *c1ccc2c(c1)COC21CN(*)C1]

(3m) One embodiment relates to embodiments (3), (3a), (3b), (3c), (3d), (3e), (3f), (3g), (3h), (3i), (3j), (3k), and (3l) wherein T₂ is

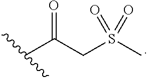 [SMILES: *C(=O)CS(C)(=O)=O]

(3n) One embodiment relates to embodiments (3), (3a), (3b), (3c), (3d), (3e), (3f), (3g), (3h), (3i), (3j), (3k), and (3l) wherein T₂ is

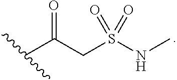 [SMILES: *C(=O)CS(=O)(=O)NC]

(3o) One embodiment relates to embodiments (3), (3a), (3b), (3c), (3d), (3e), (3f), (3g), (3h), (3i), (3j), (3k), and (3l) wherein T₂ is

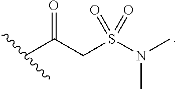 [SMILES: *C(=O)CS(=O)(=O)N(C)C]

(3p) One embodiment relates to embodiments (3), (3a), (3b), (3c), (3d), (3e), (3f), (3g), (3h), (3i), (3j), (3k), and (3l) wherein T₂ is

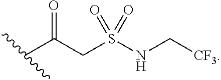 [SMILES: *C(=O)CS(=O)(=O)NCC(F)(F)F]

The following examples are intended to be illustrative and non-limiting, and represent specific embodiments of the present invention.

Analyses were performed using a Liquid Chromatography (LC) system, coupled to a quadrupole mass spectrometry (MS) detector. The UV (DAD) acquisition was performed with a scan range of 200-400 nm.

### LC-MS, Analytical Method A:

Instrument: Waters Acquity UPLC LC system—Waters SQ Detector 2; Column: Acquity UPLC BEH C18 column of 50 mm length, 2.1 mm internal diameter and 1.7 μm particle size; eluent A: water with 0.1% formic acid, eluent B: CH₃CN.

### LC-MS, Analytical Method B:

Instrument: SHIMADZU LCMS—UFLC 20-AD-LCMS 2020 MS detector; Column:

Kinetex EVO C18 2.6 μm, 50×3.0 mm; eluent A: Water/6.5 mM NH₄HCO₃, eluent B: CH₃CN.

### LC-MS, Analytical Method C:

Instrument: SHIMADZU LCMS—UFLC 20-AD-LCMS 2020 MS detector; Column: CORTECS C18 2.7 μm, 50×2.1 mm; eluent A: water+0.1 vol % formic acid, eluent B: CH₃CN+0.10 vol % formic acid.

### LC-MS, Analytical Method D:

Instrument: SHIMADZU LCMS—UFLC 20-AD-LCMS 2020 MS detector; Column: Kinetex EVO C18 2.6 μm, 50×3.0 mm; eluent A: water+0.05 vol % NH₄HCO₃, eluent B: CH₃CN.

As used herein: aq. refers to aqueous, br refers to broad, CH₃CN refers to acetonitrile, d refers to doublet, dd refers to doublet of doublet, DCM refers to dichloromethane, DIPEA refers to N-diisopropylethylamine, DMF refers to N,N-dimethylformamide, DMSO refers to dimethylsulfoxide, ee: refers to enantiomeric excess, ES refers to electrospray ionization, EtOAc refers to ethyl acetate, h refers to hour(s), HATU refers to 1-[bis(dimethylamino)methylene]-1H-1,2,3-triazolo[4,5-b]pyridinium 3-oxid hexafluorophosphate, HPLC refers to high performance liquid chromatography, iPrOH refers to isopropanol, J refers to coupling constant, LCMS refers to liquid chromatography—mass spectrometry, m/z: refers to mass-to-charge ratio, M refers to molarity, m refers to multiplet, MeOH refers to methanol, min refers to minutes, NaHCO₃refers to sodium bicarbonate, Na₂CO₃ refers to sodium carbonate, NEt₃ refers to triethylamine, NMR refers to nuclear magnetic resonance, q refers to quartet, quint refers to quintet, rt refers to room temperature, Rₜ refers to retention time, s refers to singlet, sat. refers to saturated, T refers to temperature, t refers to triplet, td refers to triplet of doublets, THF refers to tetrahydrofuran, wt refers to weight, and δ refers to chemical shift.

### rel-(R)-4-(5-(6-(difluoromethyl)-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide

### Example 1.1

and

### rel-(S)-4-(5-(6-(difluoromethyl)-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide

### Example 1.2

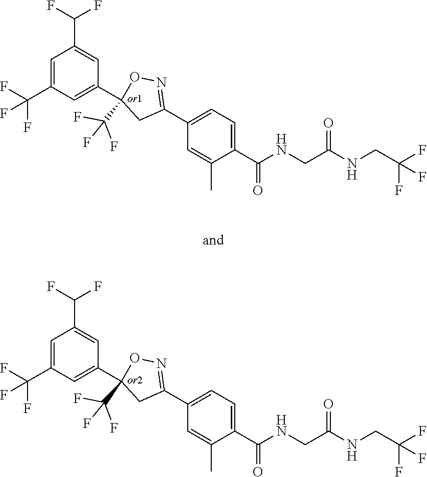 [SMILES: Cc1cc(C2=NO[C@@](c3cc(C(F)F)cc(C(F)(F)F)c3)(C(F)(F)F)C2)ccc1C(=O)NCC(=O)NCC(F)(F)F ; Cc1cc(C2=NO[C@](c3cc(C(F)F)cc(C(F)(F)F)c3)(C(F)(F)F)C2)ccc1C(=O)NCC(=O)NCC(F)(F)F]

Under nitrogen atmosphere, Pd(dppf)Cl₂ (4.55 g, 6.22 mmol) and NEt₃ (28.0 mL, 187 mmol) were added to a degassed solution of 2-bromo-4-(trifluoromethyl)pyridine (14.0 g, 62.2 mmol) in MeOH (200 mL). The resulting solution was stirred for 3 h at 80° C. under CO gas (150 psi). After completion of reaction, the mixture was filtered through Celite™ and the solvent was evaporated under reduced pressure. The crude product was purified by column chromatography on silica gel (10% EtOAc in hexanes) to afford methyl 4-(trifluoromethyl)picolinate. LC-MS: m/z=205.9 [M+H]⁺.

To a stirred solution of methyl 4-(trifluoromethyl)picolinate (11.0 g, 53.6 mmol) in CHCl₃ (120 mL) was added mCPBA (27.7 g, 161 mmol) and the resulting mixture was heated at 60ºC for 16 h. After completion of reaction, the mixture was allowed to cool down to rt and was diluted with DCM and filtered through Celite®. The filtrate was washed with sat. NaHCO₃-solution and extracted with DCM (3 x). The combined organic layers were dried over anhydrous Na₂SO₄ and concentrated under reduced pressure. The residue was purified by column chromatography on silica gel (0-15% EtOAc/petroleum ether) to afford the desired product as yellowish oil. LC-MS: m/z=222.1 [M+H]⁺.

A solution of 2-(methoxycarbonyl)-4-(trifluoromethyl)pyridine 1-oxide (8.00 g, 36.2 mmol) in POCl₃ (80 mL) was heated at 90° ° C. for 2 h. After completion of the reaction, the mixture was concentrated under reduced pressure. Ice water was added to the residue and the mixture was extracted with EtOAc (3 x). The combined organic layers were dried over anhydrous Na₂SO₄, filtered, and concentrated under reduced pressure. The crude product was purified by column chromatography on silica gel (0-15% EtOAc/petroleum ether) to afford methyl 6-chloro-4-(trifluoromethyl)picolinate as yellowish solid. LC-MS: m/z=239.9 [M+H]⁺.

To a stirred solution of methyl 6-chloro-4-(trifluoromethyl)picolinate (5.90 g, 24.6 mmol) in MeOH (8 mL) was added NaOMe (64.0 mg, 1.23 mmol) at 0° C. Then, NaBH₄ (1.86 g, 49.3 mmol) was added and the resulting reaction mixture was stirred at rt for 2 h. After completion of the reaction, the mixture was concentrated in vacuo. DCM was added and the solution was filtered. The filtrate was concentrated under reduced pressure to afford (6-chloro-4-(trifluoromethyl)pyridin-2-yl)methanol. The product was used in the next synthetic step without further purification. LC-MS: m/z=212.1 [M+H]⁺.

At 0° C., in an ice bath, Dess-Martin periodinane (22.5 g, 53.1 mmol) was added to a solution of (6-chloro-4-(trifluoromethyl)pyridin-2-yl)methanol (5.6 g, 26.5 mmol) in DCM (100 mL). The resulting reaction mixture was stirred for 2 h, where it was allowed to warm up to rt. The solvents were evaporated under reduced pressure and the residue was purified by column chromatography on silica gel (10% EtOAc in hexanes) to obtain 6-chloro-4-(trifluoromethyl)picolinaldehyde. LC-MS: m/z=209.9 [M+H]+.

To a stirred solution of 6-chloro-4-(trifluoromethyl)picolinaldehyde (2.80 g, 13.4 mmol) in DCM (100 mL) was added DAST (7.8 g, 20.0 mmol) at 0° C. The reaction mixture was stirred at rt for 2 h. After this time, the reaction was quenched by addition of ice water and the resulting aq. layer was extracted with DCM (2 x). The solvent was evaporated in vacuo to afford the desired product. 2-chloro-6-(difluoromethyl)-4-(trifluoromethyl)pyridine was used in the next synthetic step without further purification.

Under nitrogen atmosphere, Cs₂CO₃ (6.30 g, 194 mmol) was added to a degassed solution of 2-chloro-6-(difluoromethyl)-4-(trifluoromethyl)pyridine (1.50 g, 6.47 mmol) in THF (10 mL) and water (5 mL). Then, Pd(OAc)₂ (217 mg, 323 μmol), PPh₃ (203 mg, 777 μmol) and potassium trifluoro(3,3,3-trifluoroprop-1-en-2-yl)borate (1.96 g, 9.71 mmol) were added and the reaction mixture was heated at 80° C. for 16 h. After this time, the mixture was diluted with water and extracted with Et₂O (3×). The combined organic layer was dried over anhydrous Na₂SO₄, filtered and concentrated under reduced pressure. The residue was purified by column chromatography on silica gel (0-3% EtOAc/petroleum ether) to afford 2-(difluoromethyl)-4-(trifluoromethyl)-6-(3,3,3-trifluoroprop-1-en-2-yl)pyridine as brownish oil.

To a stirred solution of methyl 4-((hydroxyimino)methyl)-2-methylbenzoate (334 mg, 1.73 mmol) in DMF (3 mL) was added NCS (275 mg, 2.06 mmol) and the reaction mixture was stirred 40° C. for 10 min. After this time, the mixture was cooled down to 0° C. in an ice bath. Then, 2-(difluoromethyl)-4-(trifluoromethyl)-6-(3,3,3-trifluoroprop-1-en-2-yl)pyridine (600 mg, 2.06 mmol) in DMF (3.0 mL) and NEt₃ (417 mg, 4.12 mmol) were added and the resulting reaction mixture was allowed to stir for 16 h at rt. The mixture was then diluted with water and extracted with EtOAc (3×). The combined organic layers were dried over anhydrous Na₂SO₄ and concentrated under reduced pressure. The crude product was purified by column chromatography on silica gel (0-5% EtOAc/petroleum ether) to obtain methyl 4-(5-(6-(difluoromethyl)-4-(trifluoromethyl)pyridin-2-yl)-5-(trifluoromethyl)-4,5-dihydroisoxazol-3-yl)-2-methylbenzoate. LC-MS: m/z=483.1 [M+H]⁺.

A mixture of methyl 4-(5-(6-(difluoromethyl)-4-(trifluoromethyl)pyridin-2-yl)-5-(trifluoromethyl)-4,5-dihydroisoxazol-3-yl)-2-methylbenzoate (200 mg, 0.414 mmol) and LiOH·H₂O (520 mg, 1.24 mmol) in dioxane (2 mL) and water (2 mL) was heated at 90° C. for 6 h. The reaction was quenched by addition of aq. HCl-solution (1 M). The resulting precipitate was filtered off, washed with water, and dried in vacuo. The desired product, 4-(5-(6-(difluoromethyl)-4-(trifluoromethyl)pyridin-2-yl)-5-(trifluoromethyl)-4,5-dihydroisoxazol-3-yl)-2-methylbenzoic acid, was obtained as yellowish solid and was used in the next synthetic step without further purification. LC-MS: m/z=469.1 [M+H]⁺.

To a stirred solution of compound 4-(5-(6-(difluoromethyl)-4-(trifluoromethyl)pyridin-2-yl)-5-(trifluoromethyl)-4,5-dihydroisoxazol-3-yl)-2-methylbenzoic acid (130 mg, 277 μmol), 2-amino-N-(2,2,2-trifluoroethyl)acetamide (80.1 mg, 416 μmol), and HATU (158 mg, 416 μmol) in DMF (4 mL) was added DIPEA (107 mL, 832 μmol) and the resulting reaction mixture was stirred at rt for 2 h. The reaction mixture was quenched by addition of water and was extracted with EtOAc (3 x). The combined organic layers were washed with brine, dried over anhydrous Na₂SO₄ and concentrated under reduced pressure. The crude product was purified by column chromatography on silica gel (0-10% EtOAc/petroleum ether) to afford racemic 4-(5-(6-(difluoromethyl)-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide as a colorless solid. LC-MS (method A) Rₜ=2.22 min, m/z=607.4 [M+H]⁺. 1H NMR (DMSO-d6, 400 MHz) δ 8.61 (t, J=6.0 Hz, 2H), 8.26 (s, 1H), 8.18 (s, 1H), 7.68-7.62 (m, 2H), 7.49 (d, J=8.0 Hz, 1H), 7.16 (t, J=54 Hz, 1H), 4.50 (d, J=18 Hz, 1H), 4.35 (d, J=18 Hz, 1H), 4.00-4.87 (m, 4 H), 2.39 (s, 3H).

The two enantiomers were separated by SFC. The separation was performed on CHIRALPAK-AD-H with column dimensions of 250 mm×30 mm (5 μm), a flow rate of 100 g/min, and a CO₂-based mobile phase with 10% iPrOH containing 0.2% N,N-dimethylethylamine as additive to give Example 1.1: rel-(R)-4-(5-(6-(difluoromethyl)-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide and Example 1.2: rel-(S)-4-(5-(6-(difluoromethyl)-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide.

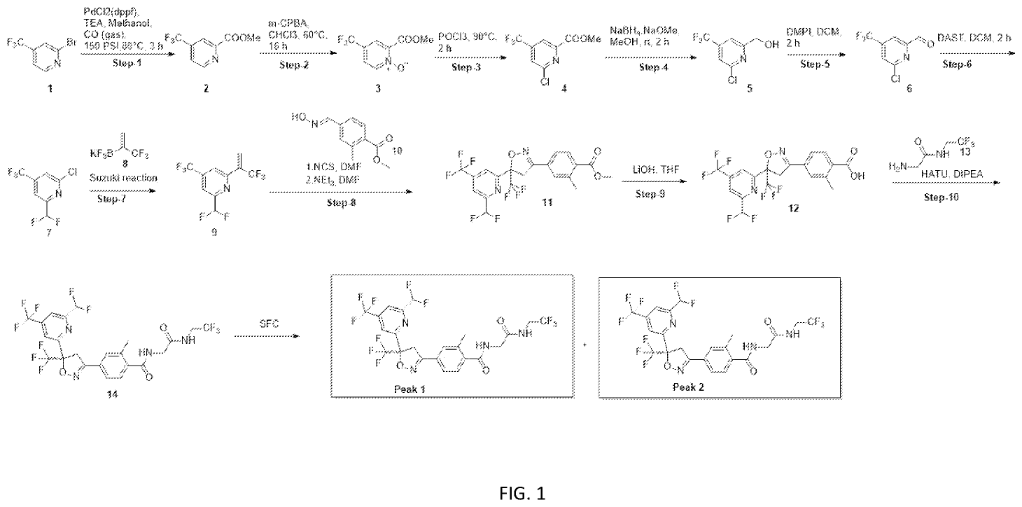

An alternate synthesis scheme for preparing Examples 1.1 and 1.2 is found in FIG. 1.

The following compounds were prepared analogously by the methodology of Examples 1.1 and 1.2:

| Ex. | Name | Structure |
|---|---|---|
| 1.3 | rel-(R)-4-(5-(6-(difluoromethyl)-4- (trifluoromethyl)pyridin-2-yl)-5- (trifluoromethyl)-4,5-dihydroisoxazol-3-yl)- 2-methyl-N-(1-(methylsulfonyl)azetidin-3- yl)benzamide | 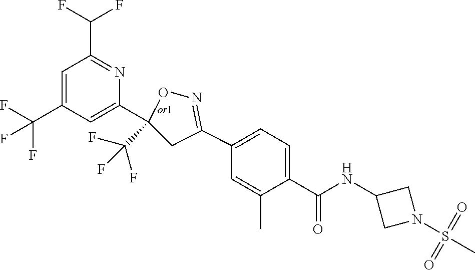 [SMILES: Cc1cc(C2=NO[C@@](c3cc(C(F)(F)F)cc(C(F)F)n3)(C(F)(F)F)C2)ccc1C(=O)NC1CN(S(C)(=O)=O)C1] |
| 1.4 | rel-(S)-4-(5-(6-(difluoromethyl)-4- (trifluoromethyl)pyridin-2-yl)-5- (trifluoromethyl)-4,5-dihydroisoxazol-3-yl)- 2-methyl-N-(1-(methylsulfonyl)azetidin-3- yl)benzamide | 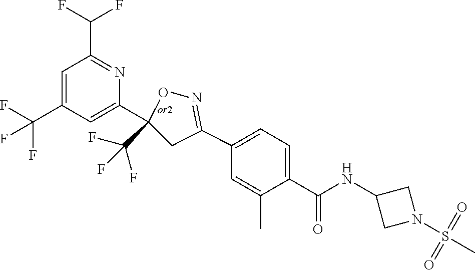 [SMILES: Cc1cc(C2=NO[C@](c3cc(C(F)(F)F)cc(C(F)F)n3)(C(F)(F)F)C2)ccc1C(=O)NC1CN(S(C)(=O)=O)C1] |
| 1.5 | rel-(R)-4-(5-(6-bromo-4- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 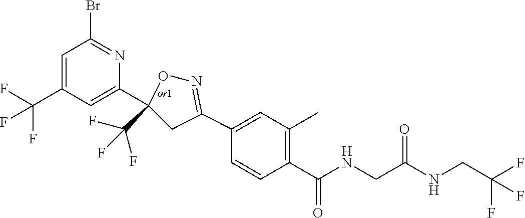 [SMILES: Cc1cc(C2=NO[C@](c3cc(C(F)(F)F)cc(Br)n3)(C(F)(F)F)C2)ccc1C(=O)NCC(=O)NCC(F)(F)F] |
| 1.6 | rel-(S)-4-(5-(6-bromo-4- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 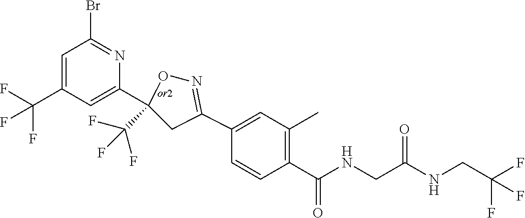 [SMILES: Cc1cc(C2=NO[C@@](c3cc(C(F)(F)F)cc(Br)n3)(C(F)(F)F)C2)ccc1C(=O)NCC(=O)NCC(F)(F)F] |
| 1.7 | rel-(R)-4-(5-(4,6- bis(trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 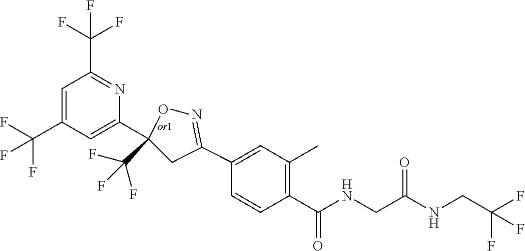 [SMILES: Cc1cc(C2=NO[C@](c3cc(C(F)(F)F)cc(C(F)(F)F)n3)(C(F)(F)F)C2)ccc1C(=O)NCC(=O)NCC(F)(F)F] |
| 1.8 | rel-(S)-4-(5-(4,6- bis(trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 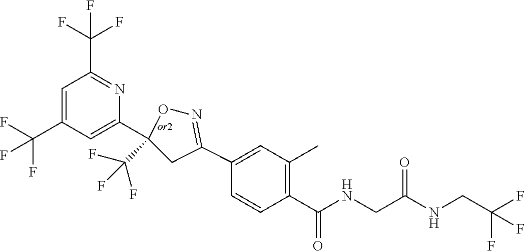 [SMILES: Cc1cc(C2=NO[C@@](c3cc(C(F)(F)F)cc(C(F)(F)F)n3)(C(F)(F)F)C2)ccc1C(=O)NCC(=O)NCC(F)(F)F] |
| 1.9 | rel-(R)-4-(5-(6-methoxy-4- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 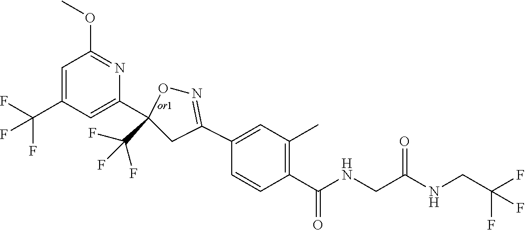 [SMILES: COc1cc(C(F)(F)F)cc([C@@]2(C(F)(F)F)CC(c3ccc(C(=O)NCC(=O)NCC(F)(F)F)c(C)c3)=NO2)n1] |
| 1.10 | rel-(S)-4-(5-(6-methoxy-4- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 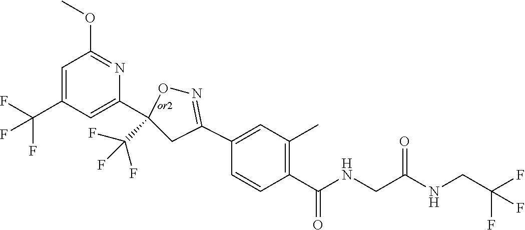 [SMILES: COc1cc(C(F)(F)F)cc([C@]2(C(F)(F)F)CC(c3ccc(C(=O)NCC(=O)NCC(F)(F)F)c(C)c3)=NO2)n1] |
| 1.11 | rel-(R)-4-(5-(4-chloro-6- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 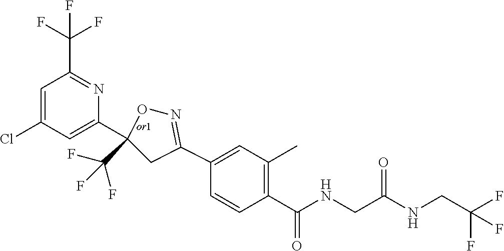 [SMILES: Cc1cc(C2=NO[C@](c3cc(Cl)cc(C(F)(F)F)n3)(C(F)(F)F)C2)ccc1C(=O)NCC(=O)NCC(F)(F)F] |
| 1.12 | rel-(S)-4-(5-(4-chloro-6- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 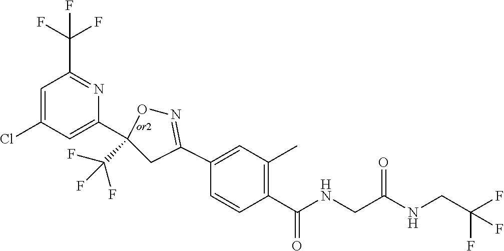 [SMILES: Cc1cc(C2=NO[C@@](c3cc(Cl)cc(C(F)(F)F)n3)(C(F)(F)F)C2)ccc1C(=O)NCC(=O)NCC(F)(F)F] |
| 1.13 | rel-(R)-4-(5-(4-chloro-6- (difluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 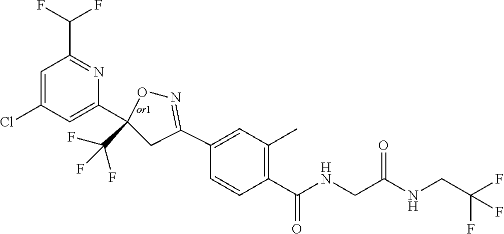 [SMILES: Cc1cc(C2=NO[C@](c3cc(Cl)cc(C(F)F)n3)(C(F)(F)F)C2)ccc1C(=O)NCC(=O)NCC(F)(F)F] |
| 1.14 | rel-(S)-4-(5-(4-chloro-6- (difluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 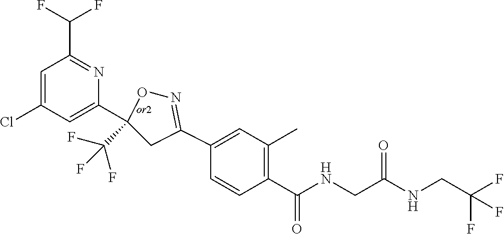 [SMILES: Cc1cc(C2=NO[C@@](c3cc(Cl)cc(C(F)F)n3)(C(F)(F)F)C2)ccc1C(=O)NCC(=O)NCC(F)(F)F] |
| 1.15 | rel-(R)-4-(5-(6-fluoro-4- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 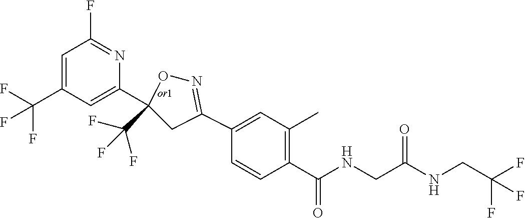 [SMILES: Cc1cc(C2=NO[C@](c3cc(C(F)(F)F)cc(F)n3)(C(F)(F)F)C2)ccc1C(=O)NCC(=O)NCC(F)(F)F] |
| 1.16 | rel-(S)-4-(5-(6-fluoro-4- (trifluoromethyl)pyridin-2-yl)-5- (trifluoromethyl)-4,5-dihydroisoxazol-3-yl)- 2-methyl-N-(2-oxo-2-((2,2,2- trifluoroethyl)amino)ethyl)benzamide | 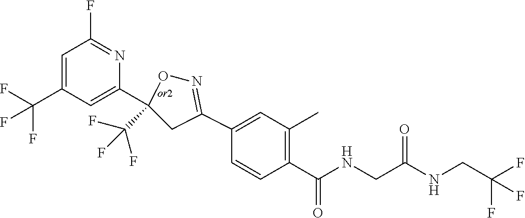 [SMILES: Cc1cc(C2=NO[C@@](c3cc(C(F)(F)F)cc(F)n3)(C(F)(F)F)C2)ccc1C(=O)NCC(=O)NCC(F)(F)F] |

### 4-((R*)-5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide

### Example 2.1 and

### 4-((S*)-5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide

### Example 2.2

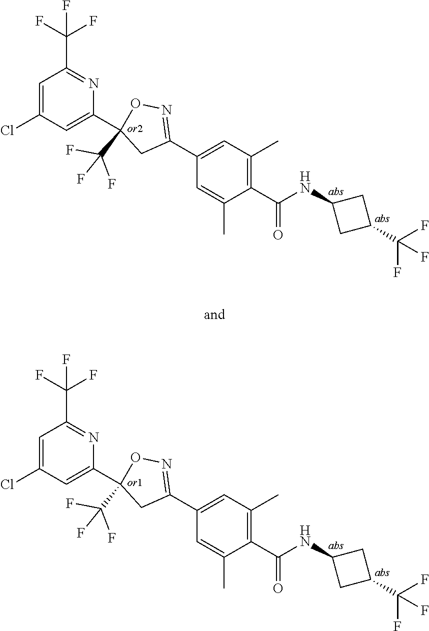 [SMILES: Cc1cc(C2=NO[C@](c3cc(Cl)cc(C(F)(F)F)n3)(C(F)(F)F)C2)cc(C)c1C(=O)N[C@H]1C[C@H](C(F)(F)F)C1 ; Cc1cc(C2=NO[C@@](c3cc(Cl)cc(C(F)(F)F)n3)(C(F)(F)F)C2)cc(C)c1C(=O)N[C@H]1C[C@H](C(F)(F)F)C1]

A mixture of 2,4-bis(chloranyl)-6-[tris(fluoranyl)methyl]pyridine (2.00 g, 9.26 mmol), trifluoro(3,3,3-trifluoroprop-1-en-2-yl)-14-borane, potassium salt (3.74 g, 18.5 mmol), Cs₂CO₃ (9.05 g, 27.8 mmol) and Pd(dppf)Cl₂ (672 mg, 926 μmol) in THF (15 mL) and water (6 mL) was stirred for 4 h under nitrogen atmosphere at 80° C. After cooling down to rt, water was added and the resulting mixture was extracted with petroleum ether. The combined organic layers were dried over anhydrous Na₂SO₄, filtered, and used directly in next step. GC-MS: Rₜ=2.43 min; m/z=275 (M)⁺.

To a solution of methyl (E)-4-((hydroxyimino)methyl)-2-methylbenzoate (2.10 g, 10.9 mmol) in DMF was added NCS (1.45 g, 10.9 mmol) at rt and the resulting mixture was stirred at rt for 30 min. Then a solution of 4-chloro-2-(trifluoromethyl)-6-(3,3,3-trifluoroprop-1-en-2-yl)pyridine in petroleum ether (10.9 mmol) was added. The mixture was stirred for 10 min and then NEt₃ (2.20 g, 21.8 mmol) was added. After 1 h, the reaction mixture poured into ice-cold water. The precipitate was collected, washed with water and then purified by column chromatography on silica gel (0-10% EtOAc in petroleum ether) to afford methyl 4-(5-(4-chloro-6-(trifluoromethyl)pyridin-2-yl)-5-(trifluoromethyl)-4,5-dihydroisoxazol-3-yl)-2-methylbenzoate as a yellow solid. LC-MS (Method C): Rₜ=1.12 min; m/z=467 (M+H)⁺.

A solution of methyl methyl 4-(5-(4-chloro-6-(trifluoromethyl)pyridin-2-yl)-5-(trifluoromethyl)-4,5-dihydroisoxazol-3-yl)-2-methylbenzoate (1.00 g, 2.14 mmol) and LiOH (256.54 mg, 10.71 mmol) in THF (15 mL) and water (15 mL) was stirred for 16 h at rt. The mixture was acidified with HCl (6 M) till pH value reached 3. The aqueous layer was extracted with EtOAc. The combined organic layers were washed with brine, dried over anhydrous Na₂SO₄, filtered and concentrated to get 4-(5-(4-chloro-6-(trifluoromethyl)pyridin-2-yl)-5-(trifluoromethyl)-4,5-dihydroisoxazol-3-yl)-2-methylbenzoic acid as a yellow solid. LC-MS (Method C): Rₜ=1.01 min; m/z=451 (M−H)⁻.

A reaction mixture of rac-4-(5-(4-chloro-6-(trifluoromethyl)pyridin-2-yl)-5-(trifluoromethyl)-4,5-dihydroisoxazol-3-yl)-2-methylbenzoic acid (214 mg, 472 μmol), trans-3-(trifluoromethyl)cyclobutan-1-amine·HCl (83.0 mg, 473 μmol), HATU (217 mg, 570 μmol) and diisopropylethylamine (367 mg, 2.84 mmol, 494 μL) was stirred for 1.5 h at rt. The resulting mixture was poured into water and then extracted with EtOAc. The combined organic layers were dried over anhydrous Na₂SO₄ and then concentrated under reduced pressure. The residue was purified by prep-TLC (PE/EA=2:1) to afford racemic 4-(5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide. LC-MS (Method D): Rₜ=1.42 min; m/z=574 (M+H)⁺. 1H-NMR (400 MHZ, DMSO-D6): δ 8.78 (d, 1H), 8.34 (s, 1H), 8.17 (s, 1H), 8.67-8.64 (m, 2H), 7.44 (d, 1H), 4.50-4.25 (m, 2H), 3.25-3.05 (m, 2H), 2.49-2.40 (m, 2H), 2.36 (s, 3H), 2.30-2.20 (m, 2H).

The two enantiomers were separated by prep-chiral-HPLC (Column: CHIRALPAK IE, 2*25 cm, 5 μm; mobile phase A: hexane (0.5% 2M NH₃-MeOH), mobile phase B: IPA) to obtain Example 2.1: 4-((R*)-5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide and Example 2.2: 4-((S*)-5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide.

The following compounds were prepared analogously by the methodology of Examples 2.1 and 2.2:

| Ex. | Name | Structure |
|---|---|---|
| 2.3 | 4-((R*)-5-(4-chloro-6- (trifluoromethyl)pyridin-2-yl)-5- (trifluoromethyl)-4,5-dihydroisoxazol-3-yl)- 2-methyl-N-((cis)-3- (trifluoromethyl)cyclobutyl)benzamide | 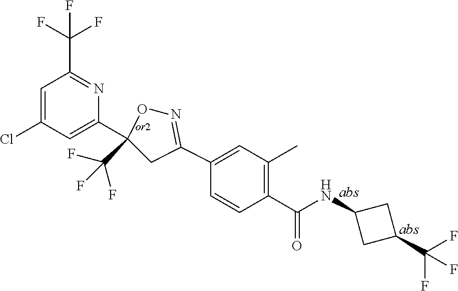 [SMILES: Cc1cc(C2=NO[C@](c3cc(Cl)cc(C(F)(F)F)n3)(C(F)(F)F)C2)ccc1C(=O)N[C@H]1C[C@@H](C(F)(F)F)C1] |
| 2.4 | 4-((S*)-5-(4-chloro-6- (trifluoromethyl)pyridin-2-yl)-5- (trifluoromethyl)-4,5-dihydroisoxazol-3-yl)- 2-methyl-N-((cis)-3- (trifluoromethyl)cyclobutyl)benzamide | 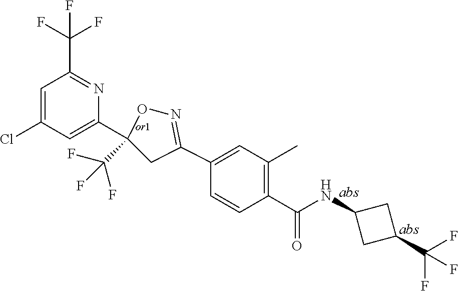 [SMILES: Cc1cc(C2=NO[C@@](c3cc(Cl)cc(C(F)(F)F)n3)(C(F)(F)F)C2)ccc1C(=O)N[C@H]1C[C@@H](C(F)(F)F)C1] |

### rel-(S)-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide

### Example 3.1

and

### rel-(R)-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide

### Example 3.2

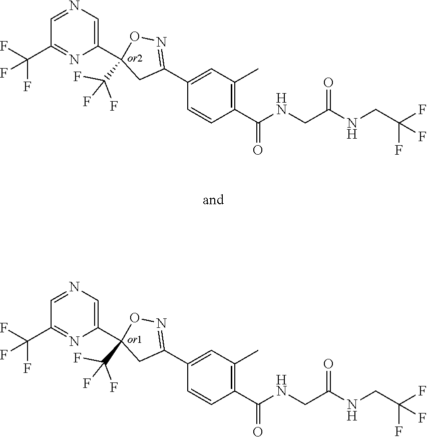 [SMILES: Cc1cc(C2=NO[C@@](c3cncc(C(F)(F)F)n3)(C(F)(F)F)C2)ccc1C(=O)NCC(=O)NCC(F)(F)F ; Cc1cc(C2=NO[C@](c3cncc(C(F)(F)F)n3)(C(F)(F)F)C2)ccc1C(=O)NCC(=O)NCC(F)(F)F]

To a solution of 2-chloranyl-6-[tris(fluoranyl)methyl]pyrazine (1.90 g, 10.4 mmol) in THF (30 mL) and water (7.5 mL) was added trifluoro(3,3,3-trifluoroprop-1-en-2-yl)-14-borane, potassium salt (3.15 g, 15.61 mmol), Cs₂CO₃ (10.2 g, 31.2 mmol) and Pd(dppf)Cl₂ (755 mg, 1.04 mmol). The reaction mixture was stirred for 24 h at 80° ° C. in seal tube under nitrogen atmosphere. The reaction mixture was quenched with water and extracted with petroleum ether. The combined organic layers were dried over anhydrous Na₂SO₄ and filtered through a pad of celite. The filtrate was concentrated under reduced pressure in ice-bath (to ˜20 mL). The solution was used in next step without any further purification. GC-MS: m/z=242 (M)⁺.

To a solution of methyl 2-methyl-4-[(E)-oxidanyliminomethyl] benzoate (1.76 g, 9.09 mmol) in DMF (30 mL) was added NCS (1.10 g, 8.26 mmol, 668 μL). The reaction mixture was stirred for 2 h at 30° C. To the above solution was added 2-[tris(fluoranyl)methyl]-6-[1-[tris(fluoranyl)methyl]vinyl] pyrazine (8.26 mmol, solution in 20 mL of petroleum ether) at 0° C. The resulting mixture was stirred for 5 min at 0° C., then NEt₃ (836 mg, 8.26 mmol, 1.15 mL) was added. The reaction mixture was warmed to rt and stirred for another 2 h. The mixture was quenched with water and extracted with EtOAc. The combined organic layers were washed with brine, dried over anhydrous Na₂SO₄. After filtration, the filtrate was concentrated under reduced pressure. The residue was purified by column chromatography on silica gel (PE/EA=4:1) to afford methyl 2-methyl-4-(5-(trifluoromethyl)-5-(6-(trifluoromethyl)pyrazin-2-yl)-4,5-dihydroisoxazol-3-yl)benzoate as yellow oil. GC-MS: m/z=433 (M)⁺.

To a solution of methyl 2-methyl-4-[(5-(trifluoromethyl)-5-[6-(trifluoromethyl)pyrazin-2-yl]-4H-isoxazol-3-yl]benzoate (400 mg, 923 μmol) in THF (4 mL) and water (4 mL) was added LiOH (111 mg, 4.62 mmol). The reaction mixture was stirred for 16 h at rt. The reaction mixture was diluted with water and acidified with HCl (2 M) till pH value reached 4. The mixture was extracted with EtOAc. The combined organic layers were dried over anhydrous Na₂SO₄. After filtration, the filtrate was concentrated under reduced pressure to afford 2-methyl-4-(5-(trifluoromethyl)-5-(6-(trifluoromethyl)pyrazin-2-yl)-4,5-dihydroisoxazol-3-yl)benzoic acid as yellow oil. LC-MS (method C): Rₜ=0.94 min; m/z=420 (M+H)⁺.

To a solution of 2-methyl-4-(5-(trifluoromethyl)-5-(6-(trifluoromethyl)pyrazin-2-yl)-4,5-dihydroisoxazol-3-yl)benzoic acid (280 mg, 668 μmol) in DMF (6 mL) was added HATU (330 mg, 868 μmol), N,N-diisopropylethylamine (345 mg, 2.67 mmol, 465 μL) and 2-azanyl-N-[2,2,2-tris(fluoranyl)ethyl]acetamide (156 mg, 1.00 mmol). The reaction mixture was stirred for 1 h at rt. The mixture was quenched with water and extracted with EtOAc. The combined organic layers were washed with brine, dried over anhydrous Na₂SO₄. After filtration, the filtrate was concentrated under reduced pressure. The residue was purified by pre-TLC (PE/EA=1:2) and prep-HPLC to afford rac-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide. LC-MS (method D): Rₜ=1.412 min; MS (ESIpos): m/z=558 (M+H)⁺. 1H NMR (400 MHZ, DMSO-d6): δ 9.39-9.36 (d, 2H), 8.63-8.60 (m, 2H), 7.68-7.66 (d, 2H), 7.50-7.48 (d, 1H), 4.54-4.50 (d, 1H), 4.36-4.31 (d, 1H), 3.99-3.91 (m, 4H), 2.40 (s, 3H).

The two enantiomers was separated by prep-chiral-HPLC (Column: CHIRALPAK IF, 2*25 cm, 5 μm; mobile phase A: hexane (0.5% 2M NH₃-MeOH), mobile phase B: EtOH) to afford Example 3.1: rel-(S)-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide and Example 3.2: rel-(R)-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide.

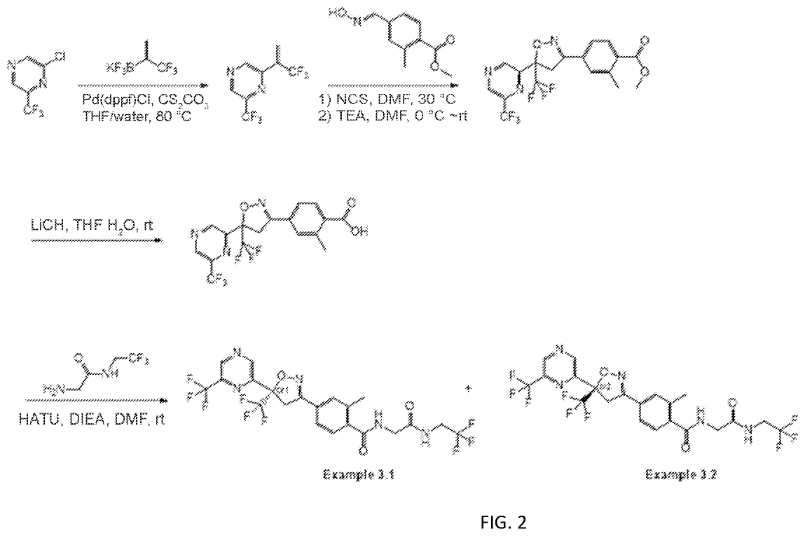

An alternate synthesis scheme for preparing Examples 3.1 and 3.2 is found in FIG. 2.

Experimental details for compounds in the tables:

| Ex. | LC-MS | ¹H NMR |
|---|---|---|
| 1.3 | LC-MS (method A) | ¹H NMR (DMSO-d6, 400 MHz) δ 9.01 (d, J = 6.8 Hz, 1 H), 8.26 |
| | Rₜ = 2.22 min, m/z = | (s, 1 H), 8.18 (s, 1 H), 7.69-7.64 (m, 2 H), 7.49 (d, J = 7.6 Hz, |
| | 601.4 [M + H]⁺ | 1 H), 7.16 (t, J = 54 Hz, 1 H), 4.71-4.61 (m, 1 H), 4.50 (d, |
| | | J = 18 Hz, 1 H), 4.35 (d, J = 18 Hz, 1 H), 4.13 (t, J = 8.0 Hz, |
| | | 2 H), 3.89 (t, J = 6.4 Hz, 2 H), 3.03 (s, 3 H), 2.38 (s, 3 H). |
| 1.4 | LC-MS (method A) | ¹H NMR (DMSO-d6, 400 MHz) δ 9.01 (d, J = 6.8 Hz, 1 H), 8.26 |
| | Rₜ = 2.22 min, m/z = | (s, 1 H), 8.18 (s, 1 H), 7.69-7.64 (m, 2 H), 7.49 (d, J = 7.6 Hz, |
| | 601.4 [M + H]⁺ | 1 H), 7.16 (t, J = 54 Hz, 1 H), 4.71-4.61 (m, 1 H), 4.50 (d, |
| | | J = 18 Hz, 1 H), 4.35 (d, J = 18 Hz, 1 H), 4.13 (t, J = 8.0 Hz, |
| | | 2 H), 3.89 (t, J = 6.4 Hz, 2 H), 3.03 (s, 3 H), 2.38 (s, 3 H). |
| 1.5 | LC-MS (method C): | ¹H-NMR (DMSO-d₆, 300 MHz): δ 8.63-8.59 (m, 2H), 8.34 (s, |
| | Rₜ = 1.26 min; m/z = | 1H), 8.00 (s, 1H), 7.67-7.64 (m, 2H), 7.49 (d, J = 8.1 Hz, 1H), |
| | 635 (M + H)⁺ | 4.48-4.27 (m, 2H), 4.01-3.89 (m, 4H), 2.39 (s, 3H). |
| 1.6 | LC-MS (method C): | ¹H-NMR (DMSO-d₆, 300 MHz): δ 8.63-8.59 (m, 2H), 8.34 (s, |
| | Rₜ = 1.26 min; m/z = | 1H), 8.00 (s, 1H), 7.67-7.64 (m, 2H), 7.49 (d, J = 8.1 Hz, 1H), |
| | 635 (M + H)⁺ | 4.48-4.27 (m, 2H), 4.00-3.89 (m, 4H), 2.39 (s, 3H). |
| 1.7 | LC-MS (method C): | ¹H-NMR (DMSO-d₆, 400 MHz): δ 8.63-8.60 (m, 2H), 8.55 (s, |
| | Rₜ = 1.24 min; m/z = | 1H), 8.31 (s, 1H), 7.68-7.65 (m, 2H), 7.49 (d, J = 8.0 |
| | 625 (M + H)⁺ | Hz, 1H), 4.53 (d, J = 18.4 Hz, 1H), 4.31 (d, J = 18.4 |
| | | Hz, 1H), 3.99-3.90 (m, 4H), 2.39 (s, 3H). |
| 1.8 | LC-MS (method C): | ¹H-NMR (DMSO-d₆, 400 MHz): δ 8.63-8.60 (m, 2H), 8.55 (s, |
| | Rₜ = 1.24 min; m/z = | 1H), 8.31 (s, 1H), 7.66 (m, 2H), 7.49 (d, J = 8.0 Hz, 1H), 4.53 |
| | 625 (M + H)⁺ | (d, J = 18.4 Hz, 1H), 4.31 (d, J = 18.4 Hz, 1H), 3.99-3.90 (m, |
| | | 4H), 2.39 (s, 3H). |
| 1.9 | LC-MS (method B) | ¹H-NMR (DMSO-d₆, 300 MHz) δ 8.62 (t, J = 6 Hz, 2 H), 7.67- |
| | Rₜ = 1.15 min, m/z = | 7.64 (m, 2 H), 7.52-7.47 (m, 2 H), 7.41 (s, 1 H), 4.38 (s, 2 H), |
| | 587.0 [M + H]⁺ | 4.01-3.91 (m, 7 H), 2.40 (s, 3 H). |
| 1.10 | LC-MS (method B) | ¹H-NMR (DMSO-d₆, 300 MHz) δ 8.64 (t, J = 6 Hz, 2 H), 7.67- |
| | Rₜ = 1.18 min, m/z = | 7.65 (m, 2 H), 7.52-7.48 (m, 2 H), 7.41 (s, 1 H), 4.39 (s, 2 H), |
| | 587.0 [M + H]⁺ | 4.01-3.91 (m, 7 H), 2.40 (s, 3 H). |
| 1.11 | LC-MS (method C): | 1H-NMR (DMSO-d₆, 400 MHz): δ 8.62 (t, 2H), 8.34 (s, 1H), |
| | Rₜ = 2.17 min; m/z = | 8.16 (s, 1H), 7.67-7.64 (m, 2H), 7.49-7.44 (d, 1H), 4.50-4.44 |
| | 591 (M + H)⁺ | (d, 1H), 4.31-4.24 (d, 1H), 4.01-3.90 (m, 4H), 2.39 (s, 3H). |
| 1.12 | LC-MS (method B): | 1H-NMR ((DMSO-d₆, 400 MHz): δ 8.642 (t, 2H), 8.34 (s, |
| | Rₜ = 1.31 min; m/z = | 1H), 8.17 (s, 1H), 7.67-7.65 (m, 2H), 7.49 (d, 1H), 4.50-4.45 |
| | 591.0 (M + H)⁺ | (d, 1H), 4.31-4.26 (d, 1H), 4.00-3.91 (m, 4H), 2.39 (s, 3H). |
| 1.13 | LC-MS (method D): | 1H-NMR (400 MHz, DMSO-d6): δ 8.63 (t, 2H), 8.04 (d, 2H), |
| | Rₜ = 1.37 min; m/z = | 7.66-7.64 (m, 2H), 7.49 (d, 1H), 7.05 (t, 1H), 4.47-4.30 (m, |
| | 573 (M + H)⁺ | 2H), 4.00-3.91 (m, 4H), 2.39 (s, 3H). |
| 1.14 | LC-MS (method D): | 1H-NMR (400 MHz, DMSO-d6): δ = 8.63 (t, 2H), 8.03 (d, |
| | Rₜ = 1.44 min; m/z = | 2H), 7.66-7.64 (m, 2H), 7.49 (d, 1H), 7.05 (t, 1H), 4.47-4.30 |
| | 573 (M + H)⁺ | (m, 2H), 4.00-3.91 (m, 4H), 2.39 (s, 3H). |
| 1.15 | LC-MS (method D): | 1H-NMR (400 MHz, DMSO-d6): δ 8.63 (t, 2H), 7.97 (d, 2H), |
| | Rₜ = 1.70 min; m/z = | 7.66-7.64 (m, 2H), 7.49 (d, 1H), 4.46-4.28 (m, 2H), 3.97-3.91 |
| | 575 (M + H)⁺ | (m, 4H), 2.39 (s, 3H). |
| 1.16 | LC-MS (method D): | 1H-NMR (300 MHz, DMSO-d6): δ 8.62 (t, 2H), 7.97 (d, 2H), |
| | Rₜ = 1.71 min; m/z = | 7.66-7.63 (m, 2H), 7.48 (d, 1H), 4.46-4.26 (m, 2H), 3.97-3.90 |
| | 575 (M + H)⁺ | (m, 4H), 2.39 (s, 3H). |
| 2.3 | LC-MS (Method D): | 1H-NMR (400 MHz, DMSO-D6): δ 8.73 (d, 1H), 8.35 (s, |
| | Rₜ = 1.40 min; m/z = | 1H), 8.16 (s, 1H), 7.66-7.63 (m, 2H), 7.42 (d, 1H), 4.50-4.22 |
| | 574 (M + H)⁺ | (m, 2H), 3.30-3.18 (m, 2H), 2.45-2.38 (m, 2H), 2.35 (s, 3H), |
| | | 2.14-2.10 (m, 2H). |
| 2.4 | LC-MS (Method D): | 1H-NMR (400 MHz, DMSO-D6): δ 8.74 (d, 1H), 8.34 (s, |
| | Rₜ = 1.40 min; m/z = | 1H), 8.17 (s, 1H), 7.66-7.63 (m, 2H), 7.41 (d, 1H), 4.50-4.24 |
| | 574 (M + H)⁺ | (m, 2H), 3.28-3.00 (m, 2H), 2.45-2.38 (m, 2H), 2.35 (s, 3H), |
| | | 2.17-2.11 (m, 2H). |

The compounds of the invention are valuable active ingredients for use in pest control. The term “pests” includes ectoparasites and endoparasites on and in animals and in the hygiene field. Particular pests are fleas, ticks, mites, flies, worms, and lice. Even more particular pests are fleas and ticks.

Animals in the context of the invention are understood to include vertebrates. The term vertebrate in this context is understood to comprise, for example fishes, amphibians, reptiles, birds, and mammals including humans. One preferred group of vertebrates according to the invention comprises warm-blooded animals including farm animals, such as cattle, horses, pigs, sheep and goats, poultry such as chickens, turkeys, guinea fowls and geese, fur-bearing animals such as mink, foxes, chinchillas, rabbits and the like, as well as companion animals such as ferrets, guinea pigs, rats, hamster, cats and dogs, and also humans. A further group of preferred vertebrates according to the invention comprises fishes including salmons.

In the context of the present invention, ectoparasites are understood to be in particular insects, acari (mites and ticks), and crustaceans (sea lice). These include insects of the following orders: Lepidoptera, Coleoptera, Homoptera, Hemiptera, Heteroptera, Diptera, Dictyoptera, Thysanoptera, Orthoptera, Anoplura, Siphonaptera, Mallophaga, Thysanura, Isoptera, Psocoptera and Hymenoptera. However, the ectoparasites which may be mentioned in particular are those which trouble humans or animals and carry pathogens, for example flies such as Musca domestica, Musca vetustissima, Musca autumnalis, Fannia canicularis, Sarcophaga carnaria, Lucilia cuprina, Lucilia sericata, Hypoderma bovis, Hypoderma lineatum, Chrysomyia chloropyga, Dermatobia hominis, Cochliomyia hominivorax, Gasterophilus intestinalis, Oestrus ovis, biting flies such as Haematobia irritans irritans, Haematobia irritans exigua, Stomoxys calcitrans, horse-flies (Tabanids) with the subfamilies of Tabanidae such as Haematopota spp. (e.g. Haematopota pluvialis) and Tabanus spp, (e.g. Tabanus nigrovittatus) and Chrysopsinae such as Chrysops spp. (e.g. Chrysops caecutiens); Hippoboscids such as Melophagus ovinus (sheep ked); tsetse flies, such as Glossinia spp; other biting insects like midges, such as Ceratopogonidae (biting midges), Simuliidae (Blackflies), Psychodidae (Sandflies); but also blood-sucking insects, for example mosquitoes, such as Anopheles spp, Aedes spp and Culex spp, fleas, such as Ctenocephalides felis and Ctenocephalides canis (cat and dog fleas), Xenopsylla cheopis, Pulex irritans, Ceratophyllus gallinae, Dermatophilus penetrans, blood-sucking lice (Anoplura) such as Linognathus spp, Haematopinus spp, Solenopotes spp, Pediculus humanis; but also chewing lice (Mallophaga) such as Bovicola (Damalinia) ovis, Bovicola (Damalinia) bovis and other Bovicola spp. Ectoparasites also include members of the order Acarina, such as mites (e.g. Chorioptes bovis, Cheyletiella spp., Dermanyssus gallinae, Ortnithonyssus spp., Demodex canis, Sarcoptes scabiei, Psoroptes ovis and Psorergates spp. and ticks. Representatives ticks are, for example, Boophilus, Amblyomma, Anocentor, Dermacentor, Haemaphysalis, Hyalomma, Ixodes, Rhipicentor, Margaropus, Rhipicephalus, Argas, Otobius and Ornithodoros and the like, which preferably infest vertebrates, for example warm-blooded animals including farm animals, such as cattle, horses, pigs, sheep and goats, poultry such as chickens, turkeys, guinea fowls, and geese, fur-bearing animals such as mink, foxes, chinchillas, rabbits and the like, as well as companion animals such as ferrets, guinea pigs, rats, hamster, cats and dogs, but also humans and fishes.

The compounds of the invention according to the invention are also active against all or individual development stages of animal pests showing normal sensitivity, as well as those showing resistance to widely used parasiticides. This is especially true for resistant insects and members of the order Acarina. The insecticidal, ovicidal and/or acaricidal effect of the active substances of the invention can manifest itself directly, i.e. killing the pests either immediately or after some time has elapsed, for example when moulting occurs, or by destroying their eggs, or indirectly, e.g. reducing the number of eggs laid and/or the hatching rate, good efficacy corresponding to a pesticidal rate (mortality) of at least 50 to 60%.

Compounds of the invention can also be used against hygiene pests, especially of the order Diptera of the families Muscidae, Sarcophagidae, Anophilidae and Culicidae; the orders Orthoptera, Dictyoptera (e.g. the family Blattidae (cockroaches), such as Blatella germanica, Blatta orientalis, Periplaneta americana) and Hymenoptera (e.g. the families Formicidae (ants) and Vespidae (wasps).

The compounds of formula (I) are also effective against ectoparasites of fishes, especially the sub-class of Copepoda (e.g. order of Siphonostomatoida (sea lice), whilst being well tolerated by fish.

The compounds of formula (I) can also be used against worms of the class Cestoda, including the subclasses Eucestoda and Cestodaria.

Compounds of the invention also have sustainable efficacy on parasitic mites and insects of plants. In the case of spider mites of the order Acarina, they are effective against eggs, nymphs and adults of Tetranychidae (Tetranychus spp. and Panonychus spp.).

They have high activity against sucking insects of the order Homoptera, especially against pests of the families Aphididae, Delphacidae, Cicadellidae, Psyllidae, Loccidae, Diaspididae and Eriophydidae (e.g. rust mite on citrus fruits); the orders Hemiptera, Heteroptera and Thysanoptera, and on the plant-eating insects of the orders Lepidoptera, Coleoptera, Diptera and Orthoptera

They are similarly suitable as a soil insecticide against pests in the soil.

The compounds of formula (I) are therefore effective against all stages of development of sucking insects and eating insects on crops such as cereals, cotton, rice, maize, soya, potatoes, vegetables, fruit, tobacco, hops, citrus, avocados and other crops.

The compounds of formula I are also effective against plant nematodes of the species Meloidogyne, Heterodera, Pratylenchus, Ditylenchus, Radopholus, Rizoglyphus etc.

The compounds of the invention are effective against helminths. Helminths are commercially important because they cause serious diseases in mammals and poultry, e.g. in sheep, pigs, goats, cattle, horses, donkeys, camels, dogs, cats, rabbits, guinea-pigs, hamsters, chicken, turkeys, guinea fowls and other farmed birds, as well as exotic birds. Typical nematodes are: Haemonchus, Trichostrongylus, Ostertagia, Nematodirus, Cooperia, Ascaris, Bunostonum, Oesophagostonum, Charbertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. The trematodes include, in particular, the family of Fasciolideae, especially Fasciola hepatica.

The pesticidal activity of the compounds of formula (I) according to the invention corresponds to a mortality rate of about 50-60% of the pests mentioned, more preferably to a mortality rate over 90%, most preferably to 95-100%. The compounds of formula (I) are preferably employed internally and externally in unmodified form or preferably together with the adjuvants conventionally used in the art of formulation and may therefore be processed in a known manner to give, for example, liquid formulations (e.g. spot-on, pour-on, spray-on, emulsions, suspensions, solutions, emulsifiable concentrates, solution concentrates), semi-solid formulations (e.g. creams, ointments, pastes, gels, liposomal preparations) and solid preparations (e.g. food additives tablets including e. g. capsules, powders including soluble powders, granules, or embeddings of the active ingredient in polymeric substances, like implants and microparticles). As with the compositions, the methods of application are selected in accordance with the intended objectives and the prevailing circumstances.

The compounds of the invention can be administered alone or in the form of a composition. In practice, the compounds of the invention are usually administered in the form of compositions, that is, in admixture with at least one acceptable excipient. The proportion and nature of any acceptable excipient(s) are determined by the properties of the selected compound of the invention, the chosen route of administration, and standard practice as in the veterinary and pharmaceutical fields.

In one embodiment, the present invention provides compositions comprising: a compound of invention and at least one acceptable excipient.

In effecting such treatment and/or control, a compound of the invention can be administered in any form and route which makes the compound bioavailable. The compounds of the invention can be administered by a variety of routes, including orally, in particularly by tablets and capsules. The compounds of the invention can be administered parenteral routes, more particularly by inhalation, subcutaneously, intramuscularly, intravenously, intraarterially, transdermally, intranasally, rectally, vaginally, occularly, topically, sublingually, and buccally, intraperitoneally, intraadiposally, intrathecally and via local delivery for example by catheter or stent. One skilled in the art can readily select the proper form and route of administration depending upon the particular characteristics of the compound selected, the disorder or condition to be treated, the stage of the disorder or condition, and other relevant circumstances. The pharmaceutical compositions of the invention may be administered to the subject, for example, in the form of tablets, including chewable tablets, capsules, cachets, papers, lozenges, wafers, elixirs, boli, ointments, transdermal patches, aerosols, inhalants, suppositories, drenches, solutions, injections, and suspensions.

The term “acceptable excipient” refers to those excipients typically used in preparing veterinary and pharmaceutical compositions and should be pure and non-toxic in the amounts used. They generally are a solid, semi-solid, or liquid material which in the aggregate can serve as a vehicle or medium for the active ingredient. Some examples of acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

In one embodiment, the composition is adapted for oral administration, such as a tablet or a capsule or a liquid formulation, for example, a solution or suspension, adapted for oral administration. In one embodiment, the composition is adapted for oral administration, such as chewable formulation, adapted for oral administration. In still another embodiment, the composition is a liquid or semi-solid formulation, for example, a solution or suspension or a paste, adapted for parenteral administration.

In one embodiment, the composition is adapted for injection administration, such as a solution or suspension, adapted for injection administration.

Particular compositions for usage on subjects in the treatment and/or control of nematodes/helminths comprise solutions; injectables; emulsions including classical emulsions, microemulsions and self-emulsifying compositions, that are waterless organic, preferably oily, compositions which form emulsions, together with body fluids, upon addition to the subject's body; suspensions (drenches); pour-on formulations; food additives; powders; tablets including effervescent tablets; boli; capsules including micro-capsules; and chewable treats. Particularly composition forms are tablets, capsules, food additives or chewable treats.

The compositions of the present invention are prepared in a manner well known in the veterinary and pharmaceutical art and include at least one of the compounds of the invention as the active ingredient. The amount of a compound of the present invention may be varied depending upon its particular form and may conveniently be between 1% to about 50% of the weight of the unit dose form. The present pharmaceutical compositions are preferably formulated in a unit dose form, each dose typically containing from about 0.5 mg to about 100 mg of a compounds of the invention. One or more unit dose form(s) may be taken to affect the treatment dosage.

In one embodiment, the present invention also provides a method for treating pests, comprising: administering to a subject in need thereof an effective amount of a compound of formula (I) or a salt thereof, the method optionally further comprising an effective amount of at least one additional active compound.

In one embodiment, the present invention also provides a method for controlling pests, comprising: administering to a subject in need thereof an effective amount of a compound of formula (I) or a salt thereof, the method optionally further comprising an effective amount of at least one additional active compound.

In one embodiment, the present invention also provides a method for treating or controlling pests, comprising: contacting a subject's environment with an effective amount of a compound of formula (I) or a salt thereof, the method optionally further comprising an effective amount of at least one additional active compound.

Thus, the invention provides for the use of the compounds of the invention as a medicament, including for the manufacture of a medicament. In one embodiment, the invention provides the manufacture of a medicament comprising a compound of formula (I) or a salt thereof for treating pests. In one embodiment, the invention provides the manufacture of a medicament comprising a compound of the invention or a salt thereof for controlling pests.

The terms “treating”, “to treat”, “treated”, or “treatment”, include without limitation restraining, slowing, stopping, reducing, ameliorating, reversing the progression or severity of an existing symptom, or preventing a disorder, condition, or disease. For example, an adult heartworm infection would be treated by administering a compound of the invention. A treatment may be applied or administered therapeutically.

The terms “control”, “controlling” or “controlled” refers to include without limitation decreasing, reducing, or ameliorating the risk of a symptom, disorder, condition, or disease, and protecting an animal from a symptom, disorder, condition, or disease. Controlling may refer to therapeutic, prophylactic, or preventative administration. For example, a larvae or immature pest may be asymptomatic but would be controlled by acting on the larvae or immature pest preventing the infection from progressing to a symptomatic or debilitating infection by mature pest.

Thus, the use of the compounds of the invention in the treatment and/or control of pests, in particular helminths, in which the endoparasitic nematodes and trematodes refers to the use of the compounds of the invention to act on the various forms of the pest throughout its life cycle, independent of whether a subject is manifesting a symptom, including morbidity or mortality, and independently of the phase(s) of the challenge.

As used herein, “administering to a subject” includes but is not limited to cutaneous, subcutaneous, intramuscular, mucosal, submucosal, transdermal, oral or intranasal administration. Administration could include injection or topical administration, for example, pour-on or spot-on administration. The pour-on or spot-on method is especially advantageous for use on herd animals such as cattle, horses, sheep or pigs, in which it is difficult or time-consuming to treat all the animals orally or by injection. Because of its simplicity, this method can of course also be used for all other animals, including individual domestic animals or pets, and is greatly favoured by the keepers of the animals, as it can often be carried out without the specialist presence of the veterinarian.

The terms “subject” and “patient” refers includes humans and non-human mammalian animals and fish, the vertebrates described herein, such as dogs, cats, mice, rats, guinea pigs, rabbits, ferrets, cows, horses, sheep, goats, and pigs. Particular subjects are mammalian pets or companion animals, such as dogs and cats and also mice, guinea pigs, ferrets, and rabbits.

The term “effective amount” refers to an amount which gives the desired benefit to the subject and includes administration for both treatment and control. The amount will vary from one individual subject to another and will depend upon a number of factors, including the overall physical condition of the subject and the severity of the underlying cause of the condition to be treated, concomitant treatments, and the amount of compound of the invention used to maintain desired response at a beneficial level.

An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, infection, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present invention, the treatment dosage, is expected to range from 0.5 mg to 100 mg. Specific amounts can be determined by the skilled person. Although these dosages are based on a subject having a mass of about 1 kg to about 20 kg, the diagnostician will be able to determine the appropriate dose for a subject whose mass falls outside of this weight range. An effective amount of the present invention, the treatment dosage, is expected to range from 0.1 mg to 10 mg/kg of the subject. The dosing regimen is expected to be monthly, quarterly, semi-annual, or annual administration.

The compounds of the invention may be combined with one or more other active compounds or therapies for the treatment of one or more disorders, diseases or conditions, including the treatment of pests, for which it is indicated. The compounds of the invention may be administered simultaneously, sequentially or separately in combination with one or more compounds or therapies for treating pests and other disorders.

Thus, it is understood that the compositions and methods of the present invention optionally include comprising an effective amount of at least one additional active compound. Additional active compounds useful in the present invention include those used to treat fleas, ticks, flies, and mosquitos and include macrocyclic lactones, like milbemycin oxime, imidacloprid, spinosad, pyriproxyfen, premethrin, S-methoprene, praziquantel and moxidectin. Further exemplary addition active compounds include, but are not limited to, afoxolaner, broflanilide, fluralaner, fluxametamide, isocycloseram, lotilaner, modoflaner, nicofluprole, sarolaner, tigolaner, albendazole, cambendazole, fenbendazole, flubendazole, mebendazole, oxfendazole, parabendazole, tiabendazole, triclabendazole, amitraz, demiditraz, clorsulon, closantel, oxyclonazide, rafoxanide, cyphenothrin, flumethrin, permethrin, cyromazine, derquantel, diamphenetide, dicyclanil, dinotefuran, imidacloprid, nitenpyram, thiamethoxam, abamectin, doramectin, emamectin, eprinomectin, ivermectin, moxidectin, selamectin, milbemycin oxime, emodepside, epsiprantel, fipronil, fluazuron, fluhexafon, indoxacarb, levamisol, lufenuron, metaflumizone, methoprene, monepantel, morantel, niclosamide, nitroscanate, nitroxynil, novaluron, oxantel, praziquantel, pyrantel, pyriprole, pyriproxyfen, sisapronil, spinosad, spinetoram and triflumezopyrim, or a salt of any of the foregoing.

The activity of the compounds of the invention may be determined by a variety of methods, including in vitro and in vivo methods.

### Example A

### In Vitro Evaluation of Ingestion Activity Against Adult Cat Fleas

To prepare the test blood mixture for feeding fleas, the test substance is dissolved in dimethyl sulphoxide and diluted with citrated cattle blood to the desired concentration. To assemble the test set-up, about 20 unfed adult male and female cat fleas (Ctenocephalides felis) are placed into a chamber which is closed at the top and bottom with gauze. A metal cylinder is sealed at one end with parafilm membrane, placed with the sealed base onto the chamber, and filled with the test blood mixture, which can be imbibed by the fleas through the parafilm membrane. The blood cylinder part of the assembled test set-up is contained at about 37° C. in an isolated air heated container above the isolating carrier plate holding the flea chambers. The flea chamber part is kept at rt. After 48 h, the insecticidal activity against fleas is determined. 100% means that all of the fleas have been killed or rendered moribund; 0% means that none of the fleas have been affected at the dose administered. From a dose response curve the respective EC50 was calculated (4-parameter logistic curve fitting). A substance shows good insecticidal activity against Ctenocephalides felis if the EC₅₀ is below an application rate of 20 ppm.

In this test for example, the following compounds from the preparation examples showed EC₅₀<1 ppm: Examples 1.1, 1.3, 1.5, 1.7, 1.9, 1.11, 1.13, 1.15, 2.1, 2.3, and 3.1.

In this test for example, the following compounds from the preparation examples showed EC50<20 ppm: Examples 1.1, 1.2, 1.3, 1.5, 1.7, 1.8, 1.9, 1.11, 1.12, 1.13, 1.14, 1.15, 1.16, 2.1, 2.2, 2.3, and 3.1.

For the data above, where single isomers were tested, without knowing the absolute configuration of the isomer, the data indicates that the test article is one isomer or another, for example, Example 1.1 or its enantiomer.

### Example B

### In Vitro Evaluation of Contact Activity Against Adult Brown Dog Ticks

In vitro contact tests with ticks are conducted with adult males and females of Rhipicephalus sanguineus. For the coating of the test vials, the test substance is dissolved and diluted in acetone p.a. to the desired concentration. The solution is then homogeneously applied to the inner walls and base of a glass vial by turning and rocking on an orbital shaker until complete evaporation of the solvent. For example, with 900 ppm solution of test substance an area-based dose of 5 μg/cm² is achieved. After the solvent is completely evaporated, 5-10 adult ticks are applied to each coated test vial, which is then sealed with a perforated plastic lid and incubated in a horizontal position in the dark at rt and ambient humidity. Acaricidal activity is determined after 48 h. For this, ticks are moved to the base of the test vial by gentle knocking, and test vials are then incubated on a hotplate at 45-50° C. for no longer than 5 min. Ticks which remain motionless on the base of the test vial or move uncoordinated without deliberately climbing up to avoid the heat are considered dead or moribund, respectively. An acaricidal activity of 100% means all ticks were dead or moribund. An acaricidal activity of 0% means none of the ticks was found dead or moribund. From a dose response curve the respective EC50 was calculated (4-parameter logistic curve fitting). A substance shows good acaricidal activity against Rhipicephalus sanguineus if the EC₅₀ is below an application rate of 5 μg/cm².

In this test for example, the following compounds from the preparation examples showed EC₅₀<0.04 μg/cm²: Examples 1.1, 1.11, 1.12 and 1.13.

In this test for example, the following compounds from the preparation examples showed EC₅₀<0.25 μg/cm²: Examples 1.1, 1.3, 1.5, 1.11, 1.12 and 1.13.

In this test for example, the following compounds from the preparation examples showed EC₅₀<5 μg/cm²: Examples 1.1, 1.2, 1.3, 1.5, 1.6, 1.7, 1.8, 1.10, 1.11, 1.12 and 1.13.

For the data above, where single isomers were tested, without knowing the absolute configuration of the isomer, the data indicates that the test article is one isomer or another, for example, Example 1.1 or its enantiomer.

### Example C

### In Vitro Evaluation of Contact Activity Against Adult Cat Fleas

In vitro contact tests with ticks are conducted with adult males and females of Ctenocephalides felis. For the coating of the test vials, the test substance is dissolved and diluted in acetone p.a. to the desired concentration. The solution is then homogeneously applied to the inner walls and base of a glass vial by turning and rocking on an orbital shaker until complete evaporation of the solvent. For example, with 900 ppm solution of test substance an area-based dose of 5 μg/cm² is achieved.

After the solvent is completely evaporated, approximately 10 adult fleas are applied to each coated test vial, which is then sealed with a perforated plastic lid and incubated in a horizontal position in the dark at rt and ambient humidity. Insecticidal activity is determined after 48 h. Fleas which remain motionless on the base of the test vial or move uncoordinated without occasionally jumping or walking straight are considered dead or moribund, respectively. An insecticidal activity of 100% means all fleas were dead or moribund. An insecticidal activity of 0% means none of the fleas was affected. From a dose response curve the respective EC50 was calculated (4-parameter logistic curve fitting). A substance shows good insecticidal activity against Ctenocephalides felis if the EC50 is below an application rate of 5 μg/cm².

In this test for example, the following compounds from the preparation examples showed EC₅₀<0.05 μg/cm²: Examples 1.1, 1.7, 1.11, 1.12 and 1.13.

In this test for example, the following compounds from the preparation examples showed EC₅₀<5 μg/cm²: Examples 1.1, 1.3, 1.5, 1.6, 1.7, 1.8, 1.9, 1.10, 1.11, 1.12 and 1.13.

For the data above, where single isomers were tested, without knowing the absolute configuration of the isomer, the data indicates that the test article is one isomer or another, for example, Example 1.1 or its enantiomer.

### Example D

### In Vitro Evaluation of Systemic Activity Against Female Engorged Cattle Ticks

To prepare the test compound mixture for injecting ticks, the test substance is dissolved in dimethyl sulphoxide and diluted with the same solvent to the desired concentration. 1 μl of the test mixture is injected into each abdomen of 5 engorged adult female cattle ticks (Rhipicephalus (Boophilus) microplus). The ticks are individually transferred into the single compartments of 5×5 replica plates and kept in a climate-controlled chamber (28° C., 85% rel. hum.). Acaricidal activity against cattle ticks is assessed after 7 days by assessment of laid fertile eggs. Eggs which do not appear normal may be stored in a climate-controlled cabinet [28° C., 85% rel h.] until larval hatch after 42 days. An acaricidal activity of 100% means that none of the ticks has laid eggs or laid eggs were infertile; 0% means that all the eggs are fertile. From a dose response curve the respective EC50 was calculated (4-parameter logistic curve fitting). A substance shows good systemic acaricidal activity against Rhipicephalus microplus if the EC50 is below an application rate of 10 μg/tick. In this test for example, the following compounds from the preparation examples showed EC₅₀<0.5 μg/tick: Examples 1.1, 1.3, 1.5, 1.7, 1.8, 1.9 1.11, and 1.13.

For the data above, where single isomers were tested, without knowing the absolute configuration of the isomer, the data indicates that the test article is one isomer or another, for example, Example 1.1 or its enantiomer.

### Example E

In vitro evaluation of contact activity against larval cattle ticks

This in vitro contact tests are conducted with larvae of Rhipicephalus microplus. For the coating of the test vials, the test substance is dissolved and diluted in acetone p.a. to the desired concentration. The solution is then homogeneously applied to the inner walls and base of a glass vial by turning and rocking on an orbital shaker until complete evaporation of the solvent. For example, with 900 ppm solution of test substance an area-based dose of 5 μg/cm² is achieved. After the solvent is completely evaporated, 10-20 larval ticks are applied to each coated test vial, which is then sealed with a perforated plastic lid and incubated in a horizontal position in trays that are kept in a climate-controlled chamber (28° C., 85% rel. hum.). Acaricidal activity is determined after 48 h. For this, vials are placed vertically and the natural negative egeotactic behavior of cattle tick larvae is used to evaluate the effects. Tick larvae which remain motionless on the base of the test vial or move uncoordinated without deliberately climbing up are considered dead or moribund, respectively. An acaricidal activity of 100% means all tick larvae were dead or moribund. An acaricidal activity of 0% means none of the tick larvae was affected. From a dose response curve the respective EC50 was calculated (4-parameter logistic curve fitting). A substance shows good acaricidal activity against Rhipicephalus sanguineus if the EC50 is below an application rate of 5 μg/cm².

In this test for example, the following compounds from the preparation examples showed EC₅₀<0.05 μg/cm²: Examples 1.1, 1.3, 1.5, 1.7, 1.9, 1.11 and 1.13.

For the data above, where single isomers were tested, without knowing the absolute configuration of the isomer, the data indicates that the test article is one isomer or another, for example, Example 1.1 or its enantiomer.

1. A compound of formula (I): 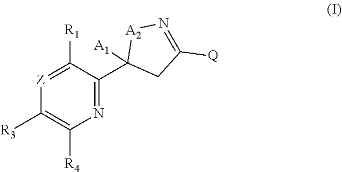
wherein
A₁ is selected from the group consisting of CF₃, CHF₂, CH₂F, and CF₂CF₃;
A₂ is O or S;
Z is N or CR₂;
R₁ is selected from the group consisting of hydrogen and halogen;
R₂ is selected from the group consisting of hydrogen, halogen, difluoromethyl, trifluoromethyl, and trifluoromethoxy;
R₃ is selected from the group consisting of hydrogen, halogen, and trifluoromethyl;
R₄ is selected from the group consisting of hydrogen, halogen, difluoromethyl, trifluoromethyl, hydroxy, methoxy, and trifluoromethoxy;
Q is selected from the group consisting of 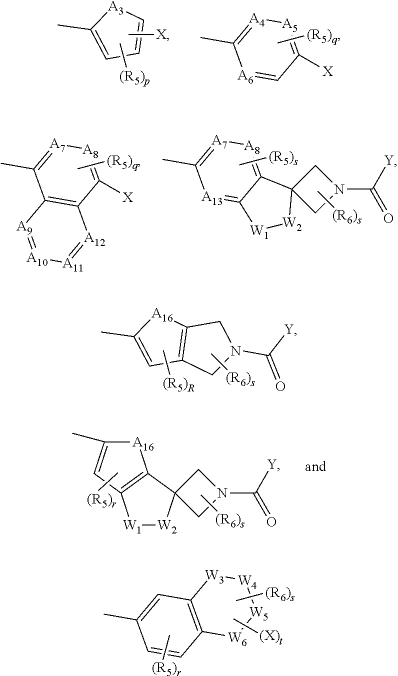
wherein
p is 0, 1, or 2;
q is 0, 1, 2, or 3;
r is 0 or 1;
s is 0, 1, or 2;
t is 0 or 1;
R₅, at each occurrence, is independently selected from the group consisting of halogen; cyano; nitro; hydroxyl; —NH₂; —NH(C₁-C₄ alkyl); —N(C₁-C₄ alkyl)₂; C₂-C₅-alkoxycarbonyl; C₁-C₆-alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl; C₁-C₆-alkoxy optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl; —NR₇C(O)(C₁-C₄ alkyl) optionally substituted on the C₁-C₄ alkyl with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂, wherein R₇ is independently selected from the group consisting of hydrogen and C₁-C₄ alkyl; —C(O)NR₇(C₁-C₄ alkyl) optionally substituted on the C₁-C₄ alkyl with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂, wherein R₇ is independently selected from the group consisting of hydrogen and C₁-C₄ alkyl; —SC₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂; and —S(O)C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), and —N(C₁-C₄ alkyl)₂;
R₆, at each occurrence, is independently selected from the group consisting of oxo, C₁-C₄ alkyl, and C₃-C₆ cycloalkyl;
A₃ is O or S;
A₄ is CH or N;
A₅ is CH or N;
A₆ is CH or N;
A₇ is CH O, S, a bond, or N;
A₈ is CH O, S, a bond, or N;
A₉ is CH or N;
A₁₀ is CH or N;
A₁₁ is CH or N;
A₁₂ is CH or N;
A₁₃ is CH or N;
A₁₄ is CH or N;
A₁₅ is CH or N;
A₁₆ is NR, O, or S, wherein R is selected from the group consisting of hydrogen, C₁-C₄ alkyl, and C₃-C₆ cycloalkyl;
W₁ is selected from the group consisting of —O—, —S—, —NR₈—, —NC(O)R₉—, —CH₂—, and ——C(O)—;
W₂ is selected from the group consisting of —O—, —S—, —NR₈—, —NC(O)R₉—, —CH₂—, and ——C(O)—;
provided that:
when W₁ is —O—, —S—, —NR₈—, or —NC(O)R₉— then W₂ is —CH₂- or ——C(O)—; and
when W₂ is —O—, —S—, —NR₈—, or —NC(O)R₉— then W₁ is —CH₂- or ——C(O)—;
W₃ is selected from the group consisting of nil, —O—, —S—, —S(O)—, —S(O)₂—, —NR₈—, —CH—, —N—, —CH₂—, and ——C(O)—;
W₄ is selected from the group consisting of nil, —O—, —S—, —S(O)—, —S(O)₂—, —NR₈-—CH—, —N—, —CH₂—, and ——C(O)—;
W₅ is selected from the group consisting of nil, —O—, —S—, —S(O)—, —S(O)₂—, —NR₈-—CH—, —N—, —CH₂—, and ——C(O)—;
W₆ is selected from the group consisting of nil, —O—, —S—, —S(O)—, —S(O)₂—, —NR₈-—CH—, —N—, —CH₂—, and ——C(O)—;
wherein the bonds between W₁, W₂, W₃, and W₄ may be single or double bonds;
provided that:
(i) not more than two of W₁, W₂, W₃, and W₄ are nil;
(ii) not more than two of W₁, W₂, W₃, and W₄ are —O—, —S—, —S(O)—, —S(O)₂—, —NR₈—, or ——C(O)—;
(iii) if two of W₁, W₂, W₃, and W₄ are —O— and/or —S— then at least one carbon atom is present between them; and
(iv) when W₁, W₂, W₃, or W₄ is —CH— and/or —NR₈— then a double bond is formed within the ring formed by W₁, W₂, W₃, and W₄;
R₈, at each occurrence, is independently selected from the group consisting of hydrogen and C₁-C₆-alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl;
R₉, at each occurrence, is independently selected from the group consisting of oxo, C₁-C₄ alkyl, and C₃-C₆ cycloalkyl;
X is 5- to 10-membered heteroaryl having 1 or 2 heteroatoms selected from the group O, S, and N,
wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, —C(O)NH—C₃-C₆ cycloalkyl, —C(O)NH—C₁-C₆ alkyl, and —C(O)NH—C₁-C₆ haloalkyl,
wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, and C₃-C₆ cycloalkyl;
or
X is selected from the group consisting of 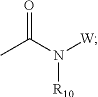
wherein
R₁₀ is selected from the group consisting of hydrogen, C₁-C₄ alkyl, C₁-C₄ haloalkyl, C₃-C₆ cycloalkyl, C₄-C₇ alkylcycloalkyl, C₂-C₇ alkylcarbonyl, C₂-C₅ alkoxycarbonyl, C₂-C₆ alkenyl, and C₂-C₆ alkynyl;
W is selected from the group consisting of
(i) hydrogen;
(ii) C₁-C₆ alkyl optionally substituted with 1 to 5 substituents independently selected from the group consisting of halogen; cyano; hydroxyl; oxo; C₁-C₄ alkoxy; C₃-C₆ cycloalkyl optionally substituted by 1 to 3 substituents independently selected from the group halogen and cyano; acetylenyl; —NH₂; C₁-C₇ aminocarbonyl; —NH(C₁-C₄ alkyl); —N(C₁-C₄ alkyl)₂; —SC₁-C₄ alkyl; —S(O)C₁-C₄ alkyl; —SO₂C₁-C₄ alkyl; —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, C₃-C₆ cycloalkyl, and —NH₂; —C(O)NH—C₁-C₆ alkyl optionally substituted with 1 to 5 substituents independently selected from the group consisting of halogen, cyano, hydroxyl, C₁-C₄ alkoxy, C₃-C₆ cycloalkyl, and —NH₂; —C(O)NH—C₁-C₆ cyanoalkyl optionally substituted with 1 to 3 halogen; —C(O)NH—C₁-C₆ haloalkyl; —C(O)-4- to 7-membered heterocycloalkyl attached by a nitrogen and optionally having 1 or 2 other heteroatoms selected from the group O, S, and N, wherein the carbons of the 4- to 7-membered heterocycloalkyl are optionally substituted with 1 to 4 substituents independently selected from the group consisting of halogen, cyano, nitro, hydroxyl, oxo, —NH₂, C₁-C₇ aminocarbonyl, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any other N in the 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, and C₁-C₄ alkyl optionally substituted with 1 to 5 substituents independently selected from the group consisting of halogen, cyano, hydroxyl, acetylenyl, C₁-C₄ alkoxy, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl; 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, C₃-C₆ cycloalkyl, C₁-C₄ haloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any S in the heteroaryl is optionally substituted with 1 or 2 oxygen atom(s); phenyl optionally substituted with 1 to 3 substituents selected from the group consisting of halogen, C₁-C₄ alkyl, cyano, and hydroxyl; C₃-C₆ cycloalkyl optionally substituted with 1 to 5 substituents independently selected from the group consisting of halogen, cyano, hydroxyl, oxo, C₁-C₄ alkoxy, C₁-C₄ alkyl optionally substituted with 1 to 3 groups selected from the group consisting of halogen and cyano, C₁-C₄ haloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, —C(O)NH—C₁-C₆ haloalkyl, C₂-C₆ alkenyl, and C₂-C₆ alkynyl; and 4- to 7-membered heterocycloalkyl having 1 or 2 heteroatoms selected from the group O, S, B, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, and C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, wherein any B in the of the 4- to 7-membered heterocycloalkyl or optionally benzo-fused 4- to 7-membered heterocycloalkyl, valency permitting, is substituted with hydroxyl, wherein any N in the 4- to 7-membered heterocycloalkyl or optionally benzo-fused 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, —C(O)—NH₂, C₁-C₄ alkyl optionally substituted with 1 to 5 substituents independently selected from the group consisting of halogen, cyano, hydroxyl, acetylenyl, C₁-C₄ alkoxy, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ haloalkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s);
(iii) C₃-C₆ cycloalkyl optionally substituted with 1 to 5 substituents independently selected from the group consisting of halogen, cyano, hydroxyl, oxo, carboxyl, C₁-C₄ alkoxy, C₁-C₄ alkyl optionally substituted with 1 to 3 groups selected from the group consisting of halogen and cyano, C₁-C₄ haloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, —C(O)NH—C₁-C₆ haloalkyl, C₂-C₆ alkenyl optionally substituted with 1 to 3 halogens, and C₂-C₆ alkynyl;
(iv) 6-membered aryl or 5- to 10-membered heteroaryl having 1, 2 or 3 heteroatoms selected from the group O, S, and N, wherein the carbons of the 6-membered aryl and the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl, wherein any N in the heteroaryl, valency permitting, is optionally substituted with a substituent selected from the group consisting of hydrogen, and C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl;
(v) 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkoxy, C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, —SO₂NH(C₁-C₄ alkyl), —SO₂N(C₁-C₄ alkyl)₂, —C(O)—NH₂, C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ haloalkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s); and
(vi) —NR₁₁R₁₂
wherein
R₁₁ is selected from the group consisting of hydrogen, C₁-C₆ alkyl, C₂-C₆ alkenyl, C₂-C₆ alkynyl, C₃-C₆ cycloalkyl, C₄-C₇ alkylcycloalkyl, C₁-C₇ alkylcarbonyl, C₁-C₇ aminocarbonyl, and C₂-C₅ alkoxycarbonyl;
R₁₂ is selected from the group consisting of hydrogen, C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl, C₃-C₆ cycloalkyl, —C(O)—C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl, 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, and C₃-C₆ cycloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s); and 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, —C(O)NH—C₃-C₆ cycloalkyl, and —C(O)NH—C₁-C₆ alkyl, wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl;
or
R₁₀ and W are taken together with the nitrogen to which they are attached to form a 4- to 7-membered ring optionally containing 1 to 2 heteroatoms selected from the group consisting of N, S, and O, wherein the carbons of the ring are optionally substituted with 1 to 4 substituents independently selected of cyano, hydroxyl, oxo, halogen, C₁-C₂ alkoxy, N,N-di-C₁-C₄-alkylaminocarboxyl, N—C₁-C₄-alkylaminocarboxyl, C₁-C₇ aminocarboxyl, C₁-C₄ alkyl optionally substituted with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, C₃-C₆ cycloalkyl optionally substituted with 1 to 3 substituents selected from the group consisting of halogen, cyano, hydroxyl, and C₁-C₄ alkoxy, —C(O)NH—C₃-C₆ cycloalkyl, —C(O)NH—C₁-C₆ alkyl, —C(O)NH—C₁-C₆ haloalkyl, 5- to 6-membered heteroaryl, and phenyl optionally substituted with 1 to 3 substituents independently selected from the group consisting of halogen, C₁-C₄ alkyl, cyano, hydroxyl, C₁-C₂ alkoxy, N, N-di-C₁-C₄-alkylaminocarboxyl, N—C₁-C₄-alkylaminocarboxyl, and C₁-C₇ aminocarboxyl, wherein any N in the 4- to 7-membered ring is substituted with a substituent selected from the group consisting of hydrogen, C₁-C₄ alkyl optionally substituted with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, C₃-C₆ cycloalkyl optionally substituted with 1 to 3 substituents independently selected from the group consisting of halogen, cyano, hydroxyl, C₁-C₄ alkoxy, —C(O)NH—C₃-C₆ cycloalkyl, and —C(O)NH—C₁-C₆ alkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered ring is optionally substituted with 1 or 2 oxygen atom(s); and
Y is C₁-C₆ alkyl optionally substituted with 1 to 5 substituents independently selected from the group consisting of halogen, cyano, hydroxyl, oxo, C₃-C₆ cycloalkyl, C₁-C₄ alkoxy, acetylenyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —SO₂NH(C₁-C₄ alkyl), —SO₂N(C₁-C₄ alkyl)₂, —SO₂NH(C₁-C₄ haloalkyl), —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, and —NH₂, —C(O)NH—C₁-C₆ alkyl, —C(O)NH—C₁-C₆ cyanoalkyl optionally substituted with 1 to 3 halogen, —C(O)NH—C₁-C₆ haloalkyl, phenyl optionally substituted with 1, 2 or 3 substituents independently selected from the group consisting of halogen, cyano, nitro, hydroxyl, C₁-C₄ alkyl optionally substituted with 1 to 3 substituents independently selected from the group consisting of halogen, cyano, hydroxyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl, and C₃-C₆ cycloalkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, C₂-C₆ alkenyl, and C₂-C₆ alkynyl, 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, C₁-C₇ aminocarbonyl, —NH(C₁-C₄ alkyl), —N(C₁-C₄ alkyl)₂, —C(O)NH—C₃-C₆ cycloalkyl, and —C(O)NH—C₁-C₆ alkyl, wherein any N in the heteroaryl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, and 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, and 5- to 6-membered heteroaryl, wherein any S in the 4- to 7-membered heterocycloalkyl or optionally benzo-fused 4- to 7-membered heterocycloalkyl is optionally substituted with 1 or 2 oxygen atom(s);
or a salt thereof.

2. A compound according to claim 1, wherein
W is selected from the group consisting of
(i) hydrogen;
(ii) C₁-C₆ alkyl optionally substituted with 1 to 5 substituents independently selected from the group consisting of halogen; cyano; hydroxyl; oxo; C₁-C₄ alkoxy; C₃-C₆ cycloalkyl optionally substituted by 1 to 3 substituents independently selected from the group halogen and cyano; acetylenyl; —NH₂; C₁-C₇ aminocarbonyl; —NH(C₁-C₄ alkyl); —N(C₁-C₄ alkyl)₂; —SC₁-C₄ alkyl; —S(O)C₁-C₄ alkyl; —SO₂C₁-C₄ alkyl; —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, C₃-C₆ cycloalkyl, and —NH₂; —C(O)NH—C₁-C₆ alkyl optionally substituted with 1 to 5 substituents independently selected from the group consisting of halogen, cyano, hydroxyl, C₁-C₄ alkoxy, C₃-C₆ cycloalkyl, and —NH₂; —C(O)NH—C₁-C₆ cyanoalkyl optionally substituted with 1 to 3 halogen; —C(O)NH—C₁-C₆ haloalkyl; —C(O)-4- to 7-membered heterocycloalkyl attached by a nitrogen and optionally having 1 or 2 other heteroatoms selected from the group O, S, and N, wherein the carbons of the 4- to 7-membered heterocycloalkyl are optionally substituted with 1 to 4 substituents independently selected from the group consisting of halogen, cyano, nitro, hydroxyl, oxo, —NH₂, C₁-C₇ aminocarbonyl, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any other N in the 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, and C₁-C₄ alkyl optionally substituted with 1 to 5 substituents independently selected from the group consisting of halogen, cyano, hydroxyl, acetylenyl, C₁-C₄ alkoxy, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl; 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, C₃-C₆ cycloalkyl, C₁-C₄ haloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any S in the heteroaryl is optionally substituted with 1 or 2 oxygen atom(s); phenyl optionally substituted with 1 to 3 substituents selected from the group consisting of halogen, C₁-C₄ alkyl, cyano, and hydroxyl; C₃-C₆ cycloalkyl optionally substituted with 1 to 5 substituents independently selected from the group consisting of halogen, cyano, hydroxyl, oxo, C₁-C₄ alkoxy, C₁-C₄ alkyl optionally substituted with 1 to 3 groups selected from the group consisting of halogen and cyano, C₁-C₄ haloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, —C(O)NH—C₁-C₆ haloalkyl, C₂-C₆ alkenyl, and C₂-C₆ alkynyl; and 4- to 7-membered heterocycloalkyl having 1 or 2 heteroatoms selected from the group O, S, B, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, and C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, wherein any B in the of the 4- to 7-membered heterocycloalkyl or optionally benzo-fused 4- to 7-membered heterocycloalkyl, valency permitting, is substituted with hydroxyl, wherein any N in the 4- to 7-membered heterocycloalkyl or optionally benzo-fused 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, —C(O)—NH₂, C₁-C₄ alkyl optionally substituted with 1 to 5 substituents independently selected from the group consisting of halogen, cyano, hydroxyl, acetylenyl, C₁-C₄ alkoxy, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ haloalkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s);
(iii) C₃-C₆ cycloalkyl optionally substituted with 1 to 5 substituents independently selected from the group consisting of halogen, cyano, hydroxyl, oxo, carboxyl, C₁-C₄ alkoxy, C₁-C₄ alkyl optionally substituted with 1 to 3 groups selected from the group consisting of halogen and cyano, C₁-C₄ haloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, —C(O)NH—C₁-C₆ haloalkyl, C₂-C₆ alkenyl optionally substituted with 1 to 3 halogens, and C₂-C₆ alkynyl;
(iv) 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl, wherein any N in the heteroaryl, valency permitting, is optionally substituted with a substituent selected from the group consisting of hydrogen, and C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl;
(v) 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, C₃-C₆ cycloalkyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, —NH₂, C₁-C₇ aminocarbonyl, —SO₂C₁-C₄ alkyl, —SO₂C₁-C₄ haloalkyl, —C(O)—NH₂, C₁-C₄ alkyl optionally substituted with 1 to 5 substituents independently selected from the group consisting of halogen, cyano, hydroxyl, acetylenyl, C₁-C₄ alkoxy, C₃-C₆ cycloalkyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ haloalkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s); and
(vi) —NR₁₁R₁₂ wherein
R₁₁ is selected from the group consisting of hydrogen, C₁-C₆ alkyl, C₂-C₆ alkenyl, C₂-C₆ alkynyl, C₃-C₆ cycloalkyl, C₄-C₇ alkylcycloalkyl, C₁-C₇ alkylcarbonyl, C₁-C₇ aminocarbonyl, and C₂-C₅ alkoxycarbonyl;
R₁₂ is selected from the group consisting of hydrogen, C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl, C₃-C₆ cycloalkyl, —C(O)—C₁-C₆ alkyl optionally substituted with 1 to 5 substituents independently selected from the group consisting of halogen, cyano, nitro, hydroxyl, oxo, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, and —SO₂C₁-C₄ alkyl, 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, and —C(O)NH—C₁-C₆ haloalkyl, and C₃-C₆ cycloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered heterocycloalkyl or optionally benzo-fused 4- to 7-membered heterocycloalkyl is optionally substituted with 1 or 2 oxygen atom(s); and 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, —C(O)NH—C₃-C₆ cycloalkyl, and —C(O)NH—C₁-C₆ alkyl, wherein any N in the heteroaryl, valency permitting, is optionally substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl;
or
R₁₀ and W are taken together with the nitrogen to which they are attached to form a 4- to 7-membered ring optionally containing 1 to 2 heteroatoms selected from the group consisting of N, S, and O, wherein the carbons of the ring are optionally substituted with 1 to 4 substituents independently selected of cyano, hydroxyl, oxo, halogen, C₁-C₂ alkoxy, N, N-di-C₁-C₄-alkylaminocarboxyl, N—C₁-C₄-alkylaminocarboxyl, C₁-C₇ aminocarboxyl, C₁-C₄ alkyl optionally substituted with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, C₃-C₆ cycloalkyl optionally substituted with 1 to 3 substituents selected from the group consisting of halogen, cyano, hydroxyl, and C₁-C₄ alkoxy, —C(O)NH—C₃-C₆ cycloalkyl, —C(O)NH—C₁-C₆ alkyl, —C(O)NH—C₁-C₆ haloalkyl, 5- to 6-membered heteroaryl, and phenyl optionally substituted with 1 to 3 substituents independently selected from the group consisting of halogen, C₁-C₄ alkyl, cyano, hydroxyl, C₁-C₂ alkoxy, N,N-di-C₁-C₄-alkylaminocarboxyl, N—C₁-C₄-alkylaminocarboxyl, and C₁-C₇ aminocarboxyl, wherein any N in the 4- to 7-membered ring is substituted with a substituent selected from the group consisting of hydrogen, C₁-C₄ alkyl optionally substituted with 1 to 5 substituents independently selected from the group consisting of halogen, cyano, hydroxyl, C₃-C₆ cycloalkyl, C₁-C₄ alkoxy, —NH₂, C₁-C₇ aminocarbonyl, —NH(C₁-C₄ alkyl), —N(C₁-C₄ alkyl)₂, and —C(O)NH—C₃-C₆ cycloalkyl, C₃-C₆ cycloalkyl optionally substituted with 1 to 3 substituents independently selected from the group consisting of halogen, cyano, hydroxyl, C₁-C₄ alkoxy, —C(O)NH—C₃-C₆ cycloalkyl, and —C(O)NH—C₁-C₆ alkyl, 5- to 6-membered heteroaryl, and phenyl optionally substituted with 1 to 3 substituents independently selected from the group consisting of halogen, C₁-C₄ alkyl, cyano, and hydroxyl, wherein any S in the 4- to 7-membered ring is optionally substituted with 1 or 2 oxygen atom(s); and
Y is C₁-C₆ alkyl optionally substituted with 1 to 5 substituents independently selected from the group consisting of halogen, cyano, hydroxyl, oxo, C₃-C₆ cycloalkyl, C₁-C₄ alkoxy, acetylenyl, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl optionally substituted with 1 to 3 substituents independently selected from the group consisting of halogen, hydroxyl, cyano, and C₁-C₄ alkyl optionally substituted with 1 to 5 substituents independently selected from the group consisting of halogen, cyano, hydroxyl, C₁-C₄ alkoxy, and —NH₂, —C(O)NH—C₁-C₆ alkyl, —C(O)NH—C₁-C₆ cyanoalkyl optionally substituted with 1 to 3 halogen, —C(O)NH—C₁-C₆ haloalkyl, phenyl optionally substituted with 1, 2 or 3 substituents independently selected from the group consisting of halogen, cyano, nitro, hydroxyl, C₁-C₄ alkyl optionally substituted with 1 to 3 substituents independently selected from the group consisting of halogen, cyano, hydroxyl, C₃-C₆ cycloalkyl, C₁-C₄ haloalkyl, C₁-C₄ alkoxy, —NH₂, —NH(C₁-C₄ alkyl), —N(C₁-C₄ alkyl)₂, and —C(O)NH—C₃-C₆ cycloalkyl, and C₃-C₆ cycloalkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, —C(O)NH—C₁-C₆ alkyl, C₂-C₆ alkenyl, and C₂-C₆ alkynyl, 5- to 10-membered heteroaryl having 1 or 2 heteroatoms selected from the group O, S, and N, wherein the carbons of the 5- to 10-membered heteroaryl are optionally substituted with 1, 2 or 3 substituents independently selected from the group consisting of halogen, cyano, nitro, hydroxyl, C₁-C₄ alkyl optionally substituted with 1 to 5 substituents independently selected from the group consisting of halogen, cyano, hydroxyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, C₁-C₄ haloalkyl, C₁-C₄ alkoxy, —NH₂, C₁-C₇ aminocarbonyl, —NH(C₁-C₄ alkyl), —N(C₁-C₄ alkyl)₂, —C(O)NH—C₃-C₆ cycloalkyl, and —C(O)NH—C₁-C₆ alkyl, wherein any N in the heteroaryl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, and 4- to 7-membered heterocycloalkyl having 1 or 2 heteroatoms selected from the group O, S, and N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1 to 4 substituents independently selected from the group consisting of halogen, cyano, nitro, hydroxyl, oxo, C₁-C₄ alkyl optionally substituted with 1 to 5 substituents independently selected from the group consisting of halogen, cyano, hydroxyl, acetylenyl, oxo, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, and C₃-C₆ cycloalkyl, wherein any N in the 4- to 7-membered heterocycloalkyl or optionally benzo-fused 4- to 7-membered heterocycloalkyl, valency permitting, is substituted with a substituent selected from the group consisting of hydrogen, C₁-C₄ alkyl optionally substituted with 1 to 5 substituents independently selected from the group consisting of halogen, cyano, hydroxyl, acetylenyl, C₁-C₄ alkoxy, —NH₂, C₁-C₇ aminocarbonyl, —NH(C₁-C₄ alkyl), —N(C₁-C₄ alkyl)₂, —SC₁-C₄ alkyl, —S(O)C₁-C₄ alkyl, —SO₂C₁-C₄ alkyl, —C(O)NH—C₃-C₆ cycloalkyl, and —C(O)NH—C₁-C₆ alkyl, C₃-C₆ cycloalkyl, and 5- to 6-membered heteroaryl, wherein any S in the 4- to 7-membered heterocycloalkyl or optionally benzo-fused 4- to 7-membered heterocycloalkyl is optionally substituted with 1 or 2 oxygen atom(s);
or a salt thereof.

3. A compound according to claim 1 wherein Z is CR₂, R₁ is hydrogen, R₂ is trifluoromethyl, R₃ is hydrogen, and R₄ is trifluoromethyl; or a salt thereof.

4. A compound according to claim 1 wherein Z is CR₂, R₁ is hydrogen, R₂ is trifluoromethyl, R₃ is hydrogen, and R₄ is bromo; or a salt thereof.

5. A compound according to claim 1 wherein Z is CR₂, R₁ is hydrogen, R₂ is trifluoromethyl, R₃ is hydrogen, and R₄ is difluoromethyl; or a salt thereof.

6. A compound according to claim 1 wherein Z is CR₂, R₁ is hydrogen, R₂ is trifluoromethyl, R₃ is hydrogen, R₄ is methoxy; or a salt thereof.

7. A compound according to claim 1 wherein Z is CR₂, R₁ is hydrogen, R₂ is trifluoromethyl, R₃ is hydrogen, R₄ is fluoro; or a salt thereof.

8. A compound according to claim 1 wherein Z is CR₂, R₁ is hydrogen, R₂ is chloro, R₅ is hydrogen, and R₄ is trifluoromethyl; or a salt thereof.

9. A compound according to claim 1 wherein Z is CR₂, R₁ is hydrogen, R₂ is chloro, R₃ is hydrogen, and R₄ is difluoromethyl; or a salt thereof.

10. A compound according to claim 1 wherein Z is CR₂, R₁ is hydrogen, R₂ is trifluoromethoxy, R₃ is hydrogen, R₄ is difluoromethyl; or a salt thereof.

11. A compound according to claim 1 wherein A₁ is CF₃; or a salt thereof.

12. A compound according to claim 1 wherein A₁ is CHF₂; or a salt thereof.

13. A compound according to claim 1, wherein Z is N.

14. A compound according to claim 1, selected from the group consisting of
rel-(S)-4-(5-(6-(difluoromethyl)-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(6-bromo-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(4,6-bis(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(4,6-bis(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(6-methoxy-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(6-(difluoromethyl)-4-(trifluoromethoxy)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(R)-4-(5-(6-bromo-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(6-bromo-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide, rel-(R)-4-(5-(4,6-bis(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(4,6-bis(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(R)-4-(5-(6-methoxy-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(6-methoxy-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(R)-4-(5-(4-chloro-6-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(4-chloro-6-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(R)-4-(5-(4-chloro-6-(difluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(4-chloro-6-(difluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(R)-4-(5-(6-fluoro-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
rel-(S)-4-(5-(6-fluoro-4-(trifluoromethyl)pyridin-2-yl)-5-(trifluoromethyl)-4,5-dihydroisoxazol-3-yl)-2-methyl-N-(2-oxo-2-((2,2,2-trifluoroethyl)amino)ethyl)benzamide,
4-((R*)-5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide,
4-((S*)-5-(4-chloro-6-(trifluoromethyl)pyridin-2-yl)-5-(trifluoromethyl)-4,5-dihydroisoxazol-3-yl)-2-methyl-N-((trans)-3-(trifluoromethyl)cyclobutyl)benzamide,
4-((R*)-5-(4-chloro-6-(trifluoromethyl)pyridin-2-yl)-5-(trifluoromethyl)-4,5-dihydroisoxazol-3-yl)-2-methyl-N-((cis)-3-(trifluoromethyl)cyclobutyl)benzamide,
4-((S*)-5-(4-chloro-6-(trifluoromethyl)pyridin-2-yl)-5-(trifluoromethyl)-4,5-dihydroisoxazol-3-yl)-2-methyl-N-((cis)-3-(trifluoromethyl)cyclobutyl)benzamide,
rel-(S)-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide,
and
rel-(R)-2-methyl-N-(2-oxo-2-((2,2,2-trifluoroethyl)amino)ethyl)-4-(5-(trifluoromethyl)-5-(6-(trifluoromethyl)pyrazin-2-yl)-4,5-dihydroisoxazol-3-yl)benzamide,
or a salt thereof.

15. A compound according to claim 1, wherein if Z is CR₂,
R₁ may be hydrogen only when R₂ is trifluoromethyl, difluoromethyl, fluoro, chloro, or bromo;
R₃ may be hydrogen only when one of R₂ or R₄ is either trifluoromethyl, difluoromethyl, fluoro, chloro, or bromo; and
at most three of R₁, R₂, R₃, and R₄ are hydrogen.

16. A compound according to claim 1, wherein if Z is CR₂,
R₁ may be hydrogen only when R₂ is trifluoromethyl, difluoromethyl, or bromo;
R₃ may be hydrogen only when one of R₂ or R₄ is either trifluoromethyl, difluoromethyl, or bromo; and
at most three of R₁, R₂, R₃, and R₄ are hydrogen.

17. A composition comprising a compound of claim 1, or a salt thereof, and at least one acceptable carrier.

18. A method of treating one or more pests comprising administering to an animal in need thereof a compound of claim 1, or a salt thereof.

19. (canceled)
